(19) United States
(12) Patent Application Publication
(10) Pub. No.: US 2025/0078267 A1
(43) Pub. Date: Mar. 6, 2025

(54) MEDICAL SUPPORT DEVICE, ENDOSCOPE APPARATUS, MEDICAL SUPPORT METHOD, AND PROGRAM

(71) Applicant: FUJIFILM CORPORATION, Tokyo (JP)

(72) Inventor: Rito MURASE, Kanagawa (JP)

(21) Appl. No.: 18/818,599

(22) Filed: Aug. 29, 2024

(30) Foreign Application Priority Data
Sep. 6, 2023 (JP) 2023-144726

(51) Int. Cl. G06T 7/00; G06V 10/74; G16H 30/20; G16H 30/40

(52) U.S. Cl. CPC G06T 7/0012; G06V 10/761; G16H 30/20; G16H 30/40; G06V 2201/032

(57) ABSTRACT

A medical support device includes a processor. The processor displays, on a display device, processing result information which is information related to a processing result of an object recognition process performed on a first medical image generated by being captured by a camera. The processor makes a display aspect of the processing result information different according to a similarity or a difference between the first medical image and a second medical image stored in a storage.

### CROSS-REFERENCE TO RELATED APPLICATIONS

This application claims priority under 35 USC 119 from Japanese Patent Application No. 2023-144726, filed on Sep. 6, 2023, the disclosure of which is incorporated by reference herein.

### BACKGROUND

### 1. Technical Field

The present disclosure relates to a medical support device, an endoscope apparatus, a medical support method, and a program.

### 2. Related Art

WO2019/130924A discloses an image processing device. The image processing device disclosed in WO2019/130924A comprises a medical image acquisition unit that acquires a medical image, a scene-of-interest recognition unit that recognizes a scene of interest from the medical image acquired using the medical image acquisition unit, a similarity calculation unit that, for the scene of interest recognized using the scene-of-interest recognition unit, calculates a similarity between the medical image acquired using the medical image acquisition unit and a standard image determined for the scene of interest, and a storage process unit that performs a process of storing the medical image in a storage device based on the similarity calculated using the similarity calculation unit.

The image processing device disclosed in WO2019/130924A comprises a medical image feature quantity extraction unit that extracts a feature quantity from the medical image, and the similarity calculation unit calculates the similarity between the medical image and the standard image based on the feature quantity of the medical image.

The image processing device disclosed in WO2019/130924A comprises a standard image acquisition unit that acquires the standard image, and a standard image feature quantity extraction unit that extracts a feature quantity from the standard image acquired using the standard image acquisition unit.

The image processing device disclosed in WO2019/130924A comprises a standard image feature quantity acquisition unit that acquires a feature quantity of the standard image, and the similarity calculation unit calculates the similarity between the medical image and the standard image based on the feature quantity of the medical image and the feature quantity of the standard image.

In the image processing device disclosed in WO2019/130924A, the scene-of-interest recognition unit recognizes a scene including a lesion as the scene of interest. In addition, the scene-of-interest recognition unit acquires a plurality of medical images from a medical image storage device in which the plurality of medical images are saved in advance, identifies a scene of interest for the plurality of medical images, and selects the standard image hardly recognized by the scene-of-interest recognition unit from among medical images not recognized as the scene of interest, and the storage process unit stores the medical image in the storage device in a case where the similarity is equal to or greater than a prescribed threshold value.

JP2023-506355A discloses a computer assisted surgery system comprising an image capturing device, a display, a user interface, and a circuit. The computer assisted surgery system disclosed in JP2023-506355A is configured such that the circuit receives information indicating a surgical scenario and a surgical process associated with the surgical scenario; acquires an artificial image of the surgical scenario; outputs the artificial image for display on the display; and receives, via the user interface, permission information indicating whether or not there is permission to execute the surgical process in a case where the surgical scenario is determined to occur.

The computer assisted surgery system disclosed in JP2023-506355A is configured such that the circuit receives a real image captured by the image capturing device; determines whether or not the real image indicates occurrence of the surgical scenario; in a case where the real image indicates the occurrence of the surgical scenario, determines whether or not there is permission to execute the surgical process; and in a case where there is permission to execute the surgical process, performs control to execute the predetermined process.

The computer assisted surgery system disclosed in JP2023-506355A is configured such that the artificial image is obtained using feature visualization of an artificial neural network configured to output information indicating the surgical scenario in a case where a real image of the surgical scenario captured by the image pickup device is input to the artificial neural network; and it is determined that the real image indicates occurrence of the surgical scenario in a case where the real image is input to the artificial neural network and the artificial neural network outputs information indicating the surgical scenario.

The computer assisted surgery system disclosed in JP2023-506355A is configured such that the circuit compares the real image to the artificial image; and executes the surgical process in a case where a similarity between the real image and the artificial image exceeds a predetermined threshold value.

In the computer assisted surgery system disclosed in JP2023-506355A, the surgical process includes adjusting a visual field of the image capturing device. In addition, the surgical scenario is one in which an object occludes the visual field of the image capturing device; and the surgical process includes adjusting the visual field of the image capturing device to avoid the occluding object. In addition, the surgical scenario is one in which the image capturing device may collide with another object; and the surgical process includes adjusting a position of the image capturing device to reduce a risk of the collision.

### SUMMARY

One embodiment according to the present disclosure provides a medical support device, an endoscope apparatus, a medical support method, and a program capable of preventing a user from recognizing an inappropriate processing result from an object recognition process on a medical image.

A first aspect according to the present disclosure is a medical support device comprising a processor, in which the processor is configured to: display, on a display device, processing result information which is information related to a processing result of an object recognition process performed on a first medical image generated by being captured by a camera; and make a display aspect of the processing result information different according to a similarity or a difference between the first medical image and a second medical image stored in a storage.

A second aspect according to the present disclosure is the medical support device according to the first aspect, in which in a case where the similarity exceeds a first threshold value or the difference is equal to or less than a second threshold value, the processing result information is displayed in a first display aspect capable of specifying that the similarity exceeds the first threshold value or the difference is equal to or less than the second threshold value, and in a case where the similarity is equal to or less than the first threshold value or the difference exceeds the second threshold value, the processing result information is displayed in a second display aspect capable of specifying that the similarity is equal to or less than the first threshold value or the difference exceeds the second threshold value.

A third aspect according to the present disclosure is the medical support device according to the second aspect, in which the second display aspect has higher visibility than the first display aspect.

A fourth aspect according to the present disclosure is the medical support device according to the third aspect, in which the processing result information is not displayed in the first display aspect, and the processing result information is displayed in the second display aspect.

A fifth aspect according to the present disclosure is the medical support device according to any one of the first to fourth aspects, in which the second medical image is an exceptional image determined as a medical image that is inappropriate for use in the object recognition process.

A sixth aspect according to the present disclosure is the medical support device according to the fifth aspect, in which the exceptional image is an image in which an imaging target that is inappropriate for use in the object recognition process is shown.

A seventh aspect according to the present disclosure is the medical support device according to the sixth aspect, in which the imaging target includes an air bubble and/or a residue.

An eighth aspect according to the present disclosure is the medical support device according to any one of the fifth to seventh aspects, in which the exceptional image is an image having a composition that is inappropriate for use in the object recognition process.

A ninth aspect according to the present disclosure is the medical support device according to the eighth aspect, in which the composition is a first composition and/or a second composition, the first composition is a composition in which a size of a target object included in an imaging range within the imaging range is larger than a first size or a composition in which the size within the imaging range is smaller than a second size, and the second composition is a composition in which the target object deviates from a center region of the imaging range.

A tenth aspect according to the present disclosure is the medical support device according to any one of the fifth to ninth aspects, in which the exceptional image is an image having an image quality that is inappropriate for use in the object recognition process.

An eleventh aspect according to the present disclosure is the medical support device according to the tenth aspect, in which the image quality includes out-of-focus and/or motion blur.

A twelfth aspect according to the present disclosure is the medical support device according to any one of the first to tenth aspects, in which the storage stores a plurality of the second medical images having different contents, and the similarity or the difference is a representative value of a plurality of comparison results obtained by comparing the first medical image with each of the plurality of second medical images, or a comparison result obtained by comparing the second medical image located at a centroid of a cluster obtained by clustering the plurality of second medical images with the first medical image.

A thirteenth aspect according to the present disclosure is the medical support device according to any one of the first to twelfth aspects, in which the processing result information includes feature region information for specifying a feature region recognized by the object recognition process, characteristic information indicating characteristics of the feature region, and/or site specifying information for specifying a site where the feature region is present.

A fourteenth aspect according to the present disclosure is the medical support device according to any one of the first to thirteenth aspects, in which the display device displays similarity-related information which is information related to the similarity, or difference-related information which is information related to the difference.

A fifteenth aspect according to the present disclosure is the medical support device according to the fourteenth aspect, in which the similarity-related information includes the similarity, information based on the similarity, the second medical image used to obtain the similarity, and/or an image based on the second medical image used to obtain the similarity, and the difference-related information includes the difference, information based on the difference, the second medical image used to obtain the difference, and/or an image based on the second medical image used to obtain the difference.

A sixteenth aspect according to the present disclosure is the medical support device according to any one of the first to fifteenth aspects, in which the camera is mounted on an endoscope and is inserted into a body to image an inside of the body.

A seventeenth aspect according to the present disclosure is the medical support device according to any one of the first to sixteenth aspects, in which the object recognition process is a process of recognizing a lesion.

An eighteenth aspect according to the present disclosure is the medical support device according to any one of the first to seventeenth aspects, in which the processing result is generated by a trained model by inputting the first medical image to the trained model.

A nineteenth aspect according to the present disclosure is a medical support device comprising a processor, in which in a case where a similarity between a first medical image generated by being captured by a camera and a second medical image stored in a storage exceeds a first threshold value or a difference between the first medical image and the second medical image is equal to or less than a second threshold value, the processor does not execute an object recognition process on a first medical image, and in a case where the similarity is equal to or less than the first threshold value or the difference exceeds the second threshold value, the processor executes the object recognition process.

A twentieth aspect according to the present disclosure is an endoscope apparatus comprising: the medical support device according to any one of the first to nineteenth aspects; and the camera.

A twenty-first aspect according to the present disclosure is a medical support method comprising: displaying, on a display device, processing result information which is information related to a processing result of an object recognition process performed on a first medical image generated by being captured by a camera; and making a display aspect of the processing result information different according to a similarity or a difference between the first medical image and a second medical image stored in a storage.

A twenty-second aspect according to the present disclosure is a program for causing a computer to execute a process comprising: displaying, on a display device, processing result information which is information related to a processing result of an object recognition process performed on a first medical image generated by being captured by a camera; and making a display aspect of the processing result information different according to a similarity or a difference between the first medical image and a second medical image stored in a storage.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope apparatus is used by a doctor;

FIG. 2 is a conceptual diagram showing an example of an overall configuration of the endoscope apparatus;

FIG. 3 is a block diagram showing an example of a hardware configuration of an electric system of the endoscope apparatus;

FIG. 4 is a block diagram showing an example of functions of main units of a processor included in a medical support device and an example of information stored in a storage;

FIG. 5 is a conceptual diagram showing an example of a content of an exceptional image set;

FIG. 6 is a conceptual diagram showing an example of processing contents of a recognition unit and an example of an aspect around the recognition unit;

FIG. 7 is a conceptual diagram showing an example of processing contents of a determination unit and an example of an aspect around the determination unit;

FIG. 8 is a conceptual diagram showing a screen example in a case where processing result information is displayed on a screen and a screen example in a case where the processing result information is not displayed on the screen;

FIG. 9 is a flowchart showing an example of a flow of a medical support process;

FIG. 10 is a conceptual diagram showing an example of a screen example in a case where the processing result information is displayed on a screen regardless of whether or not a similarity is equal to or less than a first threshold value;

FIG. 11 is a conceptual diagram showing a modification example of the processing contents of the determination unit and an example of an aspect around the determination unit;

FIG. 12 is a conceptual diagram showing an example of difference-related information and the like displayed in a second display region in a case where a difference is equal to or less than a second threshold value;

FIG. 13 is a conceptual diagram showing an example of the processing contents of the determination unit and an example of an aspect around the determination unit in a case where the determination unit compares a representative similarity with a first threshold value;

FIG. 14 is a conceptual diagram showing an example of the processing contents of the determination unit in a case where the determination unit compares an exceptional image acquired from a centroid of a cluster with a frame;

FIG. 15 is a conceptual diagram showing an example of processing contents in a case where a recognition process is controlled according to a determination result by the determination unit; and

FIG. 16 is a conceptual diagram showing an example of a series of pieces of processing in which a processor included in a computer gives a processing execution request to an external device via a network, the external device executes processing according to the processing execution request, and the processor included in the computer receives a processing result from the external device.

### DETAILED DESCRIPTION

Hereinafter, examples of embodiments of a medical support device, an endoscope apparatus, a medical support method, and a program according to the present disclosure will be described with reference to the accompanying drawings.

First, the wording used in the following description will be described.

CPU is an abbreviation for a “central processing unit”. GPU is an abbreviation for a “graphics processing unit”. GPGPU is an abbreviation for “general-purpose computing on graphics processing units”. APU is an abbreviation for an “accelerated processing unit”. TPU is an abbreviation for a “tensor processing unit”. RAM is an abbreviation for a “random access memory”. NVM is an abbreviation for a “non-volatile memory”. EEPROM is an abbreviation for an “electrically erasable programmable read-only memory”. ASIC is an abbreviation for an “application specific integrated circuit”. PLD is an abbreviation for a “programmable logic device”. FPGA is an abbreviation for a “field-programmable gate array”. SoC is an abbreviation for a “system-on-a-chip”. SSD is an abbreviation for a “solid state drive”. USB is an abbreviation for a “universal serial bus”. HDD is an abbreviation for a “hard disk drive”. EL is an abbreviation for “electro-luminescence”. CMOS is an abbreviation for a “complementary metal oxide semiconductor”. CCD is an abbreviation for a “charge coupled device”. AI is an abbreviation for “artificial intelligence”. BLI is an abbreviation for “blue light imaging”. LCI is an abbreviation for “linked color imaging”. I/F is an abbreviation for an “Interface”. SSL stands for a “sessile serrated lesion”. LAN is an abbreviation for a “local area network”. WAN is an abbreviation for a “wide area network”. 5G is an abbreviation for a “5th generation mobile communication system”.

In the following description, a processor with a reference (hereinafter, simply referred to as a “processor”) may be one computing device or a combination of a plurality of computing devices. In addition, the processor may be one type of a computing device or a combination of a plurality of types of computing devices. Examples of the computing device include a CPU, a GPU, a GPGPU, an APU, or a TPU.

In the following description, a memory with a reference is a memory such as a RAM in which information is temporarily stored, and is used as a work memory by the processor.

In the following description, a storage with a reference is one or a plurality of non-volatile storage devices that store various programs, various parameters, and the like. Examples of the non-volatile storage device include a flash memory, a magnetic disk, or a magnetic tape. In addition, other examples of the storage include a cloud storage.

In the following embodiment, an external I/F with a reference transmits and receives various types of information between a plurality of devices connected to each other. Examples of the external I/F include a USB interface. A communication I/F including a communication processor, an antenna, and the like may be applied to the external I/F. The communication I/F performs communication between a plurality of computers. Examples of a communication standard applied to the communication I/F include a wireless communication standard including 5G, Wi-Fi (registered trademark), or Bluetooth (registered trademark).

In the following embodiments, “A and/or B” is synonymous with “at least one of A or B”. That is, “A and/or B” means that it may be only A, only B, or a combination of A and B. In addition, in the present specification, in a case where three or more matters are associated and represented by “and/or”, the same concept as “A and/or B” is applied.

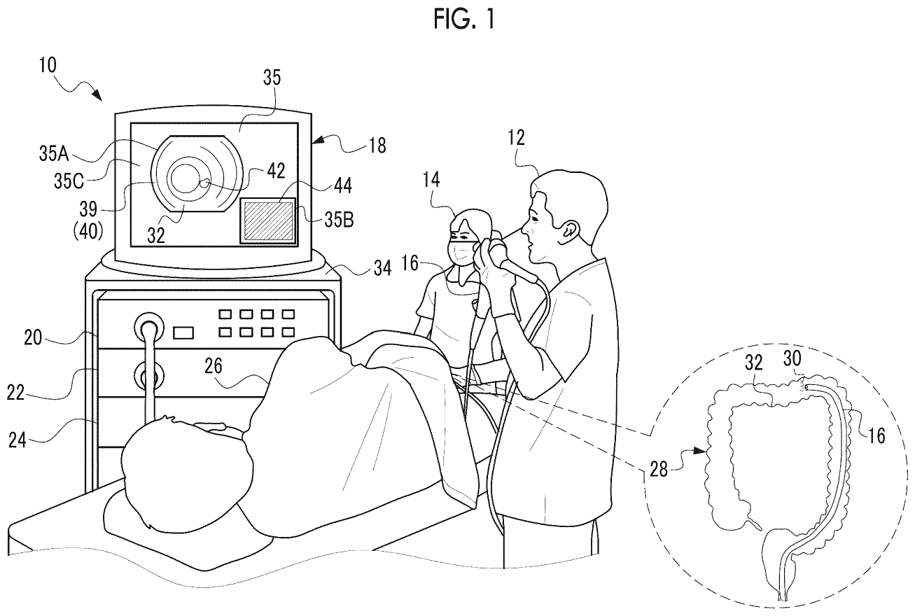

FIG. 1 is a conceptual diagram showing an example of an aspect in which an endoscope apparatus 10 is used. As shown in FIG. 1, the endoscope apparatus 10 is used by a doctor 12 in an endoscopy or the like. The endoscopy is assisted by a staff such as a nurse 14.

The endoscope apparatus 10 is connected to a communication device (not shown) in a communicable manner, and information obtained by the endoscope apparatus 10 is transmitted to the communication device. Examples of the communication device include a server, a personal computer, and/or a tablet terminal that manage various types of information such as an electronic medical record. The communication device receives the information transmitted from the endoscope apparatus 10 and executes a process using the received information (for example, a process of storing the information in an electronic medical record or the like).

The endoscope apparatus 10 comprises an endoscope 16, a display device 18, a light source device 20, a control device 22, and a medical support device 24. In the present embodiment, the endoscope apparatus 10 is an example of an “endoscope apparatus” according to the present disclosure, and the display device 18 is an example of a “display device” according to the present disclosure.

The endoscope apparatus 10 is a modality for performing medical care on a large intestine 28 included in a body of a subject 26 (for example, a patient) by using the endoscope 16. In the present embodiment, the large intestine 28 is a target to be observed by the doctor 12.

The endoscope 16 is used by the doctor 12 and is inserted into the body of the subject 26. In the present embodiment, the endoscope 16 is inserted into the large intestine 28 which is a luminal organ of the subject 26.

The endoscope apparatus 10 causes the endoscope 16 inserted into the large intestine 28 of the subject 26 to image the inside of the large intestine 28 of the subject 26 and performs various medical treatments on the large intestine 28 as necessary.

The endoscope apparatus 10 acquires and outputs an image showing an aspect in the large intestine 28 by imaging the inside of the large intestine 28 of the subject 26. In the present embodiment, the endoscope apparatus 10 is an endoscope apparatus having an optical imaging function of capturing reflected light obtained by being reflected by an intestinal wall 32 of the large intestine 28 by irradiating the inside of the large intestine 28 with light 30.

Although the endoscopy of the large intestine 28 is illustrated here, this is merely an example, and the present disclosure is established even in a case of an endoscopy of a luminal organ such as an esophagus, a stomach, a duodenum, or a trachea.

The light source device 20, the control device 22, and the medical support device 24 are installed on a wagon 34. A plurality of tables are provided in the wagon 34 along a vertical direction, and the medical support device 24, the control device 22, and the light source device 20 are installed from a lower table to an upper table. In addition, the display device 18 is installed on the uppermost table in the wagon 34.

The control device 22 controls the entire endoscope apparatus 10. The medical support device 24 performs various types of image processing on an image obtained by imaging the intestinal wall 32 with the endoscope 16 under the control of the control device 22.

The display device 18 displays various types of information including the image. Examples of the display device 18 include a liquid crystal display or an EL display. In addition, a tablet terminal with a display may be used instead of the display device 18 or together with the display device 18.

A screen 35 is displayed on the display device 18. The screen 35 includes a plurality of display regions. The plurality of display regions are arranged side by side in the screen 35. In the example shown in FIG. 1, as an example of the plurality of display regions, a first display region 35A, a second display region 35B, and a third display region 35C are shown. A size of the first display region 35A is larger than a size of the second display region 35B. The first display region 35A is used as a main display region, and the second display region 35B is used as a sub-display region. A size relationship between the first display region 35A and the second display region 35B is not limited to this, and need only be a size relationship that fits on the screen 35. The third display region 35C is a display region other than the first display region 35A and the second display region 35B in the screen 35, and is used as a sub-display region.

An endoscopic moving image 39 is displayed in the first display region 35A. The endoscopic moving image 39 is a moving image acquired by imaging the intestinal wall 32 with the endoscope 16 inside the large intestine 28 of the subject 26. In the example shown in FIG. 1, as an example of the endoscopic moving image 39, a moving image in which the intestinal wall 32 is shown is shown.

The intestinal wall 32 shown in the endoscopic moving image 39 includes a lesion 42 (for example, one lesion 42 in the example shown in FIG. 1,) as a region of interest (that is, an observation target region) that is watched by the doctor 12, and the doctor 12 can visually recognize an aspect of the intestinal wall 32 including the lesion 42 through the endoscopic moving image 39. In the present embodiment, the lesion 42 is an example of a “feature region” and a “lesion” according to the present disclosure.

The lesion 42 has various types, and examples of the type of the lesion 42 include a neoplastic polyp and a non-neoplastic polyp. Examples of the type of the neoplastic polyp include an adenomatous polyp (for example, SSL). Examples of the type of the non-neoplastic polyp include a hamartomatous polyp, a hyperplastic polyp, and an inflammatory polyp. The types illustrated here are types assumed in advance as the types of the lesion 42 in a case where the endoscopy is performed on the large intestine 28, and the types of the lesion 42 may be different depending on the organ on which the endoscopy is performed.

In the present embodiment, for convenience of description, a form example is described in which one lesion 42 is shown in the endoscopic moving image 39, but the present disclosure is not limited to this. The present disclosure is established even in a case where a plurality of the lesions 42 are shown in the endoscopic moving image 39.

In the present embodiment, the lesion 42 is illustrated, but this is merely an example. The region of interest (that is, the observation target region) that is watched by the doctor 12 may be a feature region having some unique feature, such as an organ (for example, a duodenal papilla), a mark, an artificial treatment tool (for example, an artificial clip), a treated region (for example, a region in which a trace of removal of a polyp or the like remains), or the like.

The image displayed in the first display region 35A is one frame 40 included in a moving image configured to include a plurality of frames 40 along a time series. That is, a plurality of frames 40 along the time series are displayed in the first display region 35A at a predetermined frame rate (for example, several tens of frames/second).

Examples of the moving image displayed in the first display region 35A include a moving image of a live view method. The live view method is only an example, and a moving image which is temporarily stored in a memory or the like and then is displayed, such as a moving image of a post view method, may be employed. In addition, each frame included in a recording moving image stored in a memory or the like may be reproduced and displayed on the screen 35 (for example, the first display region 35A) as the endoscopic moving image 39.

In the screen 35, the second display region 35B is adjacent to the first display region 35A and is displayed in the lower right in the screen 35 in front view. A display position of the second display region 35B may be anywhere in the screen 35 of the display device 18. However, the display position is preferably displayed at a position comparable to the endoscopic moving image 39.

In the second display region 35B, assistance information 44 for assisting the doctor 12 in medical determination or the like in the endoscopy is displayed. The assistance information 44 is information referred to by the doctor 12. Examples of the assistance information 44 include various types of information about the subject 26 in which the endoscope 16 is inserted into the body, and/or various types of information obtained by performing a medical support process described below.

Visible information obtained by performing processing using AI on the frame 40 is displayed in the third display region 35C. Details thereof will be described later.

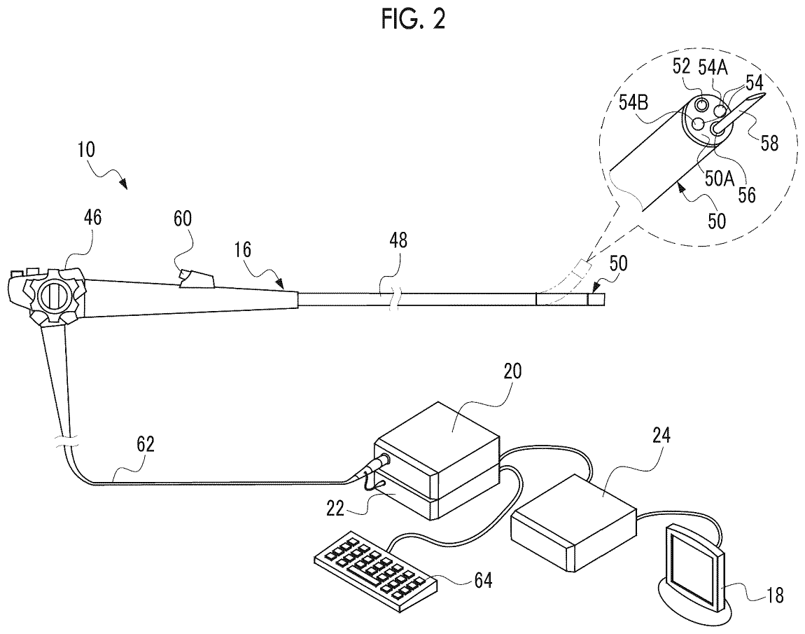

FIG. 2 is a conceptual diagram showing an example of an overall configuration of the endoscope apparatus 10. As shown in FIG. 2, the endoscope 16 comprises an operating part 46 and an insertion part 48. The insertion part 48 is partially bent by operating the operating part 46. The insertion part 48 is inserted into the large intestine 28 while being bent according to a shape of the large intestine 28 (see FIG. 1) in response to the operation of the operating part 46 by the doctor 12 (see FIG. 1).

A camera 52, an illumination device 54, and a treatment tool opening 56 are provided in a distal end part 50 of the insertion part 48. The camera 52 and the illumination device 54 are provided on a distal end surface 50A of the distal end part 50. Here, although a form example is described in which the camera 52 and the illumination device 54 are provided on the distal end surface 50A of the distal end part 50, this is merely an example. The camera 52 and the illumination device 54 may be provided on a side surface of the distal end part 50, so that the endoscope 16 may be configured as a side-viewing endoscope.

The camera 52 is mounted on the endoscope 16 and is inserted into a body cavity of the subject 26 to image the observation target region to generate the frame 40. In the present embodiment, the camera 52 generates the endoscopic moving image 39 including the plurality of frames 40 along the time series by imaging the inside of the body (for example, the inside of the large intestine 28) of the subject 26. Examples of the camera 52 include a CMOS camera. However, this is only an example, and the camera may be other types of camera such as a CCD camera. In the present embodiment, the camera 52 is an example of a “camera” according to the present disclosure, and the frame 40 is an example of a “first medical image” according to the present disclosure.

The illumination device 54 has illumination windows 54A and 54B. The illumination device 54 emits the light 30 (see FIG. 1) through illumination windows 54A and 54B. Examples of the type of the light 30 emitted from the illumination device 54 include visible light (for example, white light) and invisible light (for example, near-infrared light). In addition, the illumination device 54 emits special light through the illumination windows 54A and 54B. Examples of the special light include light for BLI and/or light for LCI. The camera 52 images the inside of the large intestine 28 by an optical method in a state in which the light 30 is emitted inside the large intestine 28 by the illumination device 54.

The treatment tool opening 56 is an opening through which a treatment tool 58 protrudes from the distal end part 50. In addition, the treatment tool opening 56 is also used as a suction port for sucking blood, internal filth, and the like and a delivery port for sending out a fluid.

A treatment tool insertion port 60 is formed in the operating part 46, and the treatment tool 58 is inserted into the insertion part 48 through the treatment tool insertion port 60. The treatment tool 58 passes through the insertion part 48 and protrudes from the treatment tool opening 56 to the outside. In the example shown in FIG. 2, an aspect is shown in which a puncture needle protrudes from the treatment tool opening 56 as the treatment tool 58. Here, the puncture needle is illustrated as the treatment tool 58, but this is merely an example. The treatment tool 58 may be a grasping forceps, a papillotomy knife, a snare, a catheter, a guide wire, a cannula, and/or a puncture needle with a guide sheath, or the like.

The endoscope 16 is connected to the light source device 20 and the control device 22 via a universal cord 62. The medical support device 24 and a reception device 64 are connected to the control device 22. In addition, the display device 18 is connected to the medical support device 24. That is, the control device 22 is connected to the display device 18 via the medical support device 24.

Here, since the medical support device 24 is illustrated as an externally connected device for expanding a function performed by the control device 22, a form example is described in which the control device 22 and the display device 18 are indirectly connected to each other via the medical support device 24, but this is merely an example. For example, the display device 18 may be directly connected to the control device 22. In this case, for example, the functions of the medical support device 24 may be provided in the control device 22, or the control device 22 may be provided with a function of causing a server (not shown) to execute the same process as the process (for example, a medical support process which will be described below) executed by the medical support device 24, receiving a processing result of the server, and using the processing result.

The reception device 64 receives an instruction from the doctor 12 and outputs the received instruction as an electric signal to the control device 22. Examples of the reception device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light to the illumination device 54. A light guide is provided in the illumination device 54, and the light supplied from the light source device 20 is emitted from the illumination windows 54A and 54B through the light guide. The control device 22 causes the camera 52 to perform imaging, acquires the endoscopic moving image 39 (see FIG. 1) from the camera 52, and outputs the endoscopic moving image 39 to a predetermined output destination (for example, the medical support device 24).

The medical support device 24 supports medical care (here, as an example, an endoscopy) by performing various types of image processing on the endoscopic moving image 39 input from the control device 22. The medical support device 24 outputs the endoscopic moving image 39 that has been subjected to various types of image processing to a predetermined output destination (for example, the display device 18).

Here, a form example is described in which the endoscopic moving image 39 output from the control device 22 is output to the display device 18 via the medical support device 24, but this is merely an example. For example, the control device 22 and the display device 18 may be connected to each other, and the endoscopic moving image 39 that has been subjected to the image processing by the medical support device 24 may be displayed on the display device 18 via the control device 22.

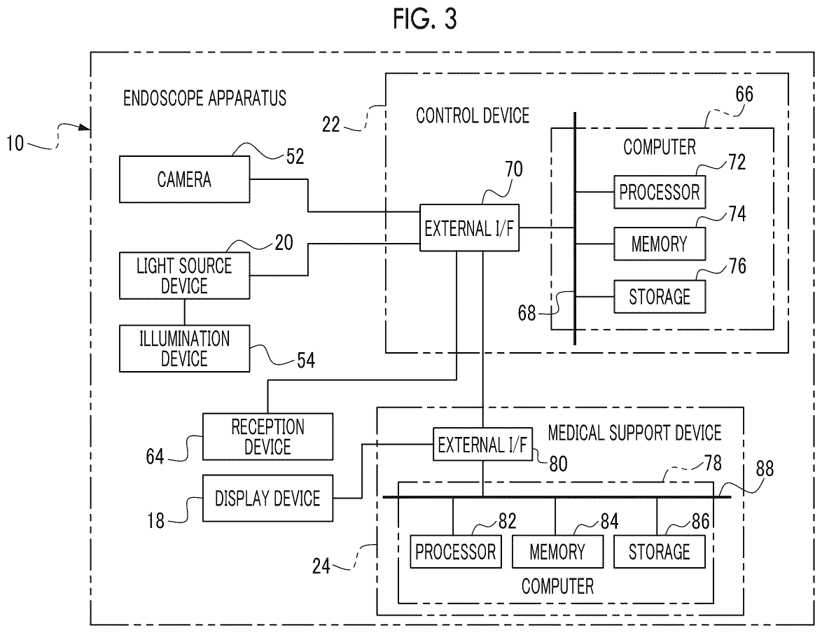

FIG. 3 is a block diagram showing an example of a hardware configuration of an electric system of the endoscope apparatus 10. As shown in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as “first external devices”) outside the control device 22 and the processor 72.

As one of the first external devices, the camera 52 is connected to the external I/F 70, and the external I/F 70 transmits and receives various types of information between the camera 52 and the processor 72. The processor 72 controls the camera 52 via the external I/F 70. In addition, the processor 72 acquires the endoscopic moving image 39 (see FIG. 1) obtained by imaging the inside of the large intestine 28 (see FIG. 1) by the camera 52 via the external I/F 70.

As one of the first external devices, the light source device 20 is connected to the external I/F 70, and the external I/F 70 transmits and receives various types of information between the light source device 20 and the processor 72. The light source device 20 supplies light to the illumination device 54 under the control of the processor 72. The illumination device 54 performs irradiation with the light supplied from the light source device 20.

As one of the first external devices, the reception device 64 is connected to the external I/F 70. The processor 72 acquires the instruction received by the reception device 64 via the external T/F 70 and performs a process corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the present embodiment, the medical support device 24 is an example of a “medical support device” according to the present disclosure, the computer 78 is an example of a “computer” according to the present disclosure, and the processor 82 is an example of a “processor” according to the present disclosure.

Since a hardware configuration (that is, the processor 82, the memory 84, and the storage 86) of the computer 78 is basically the same as the hardware configuration of the computer 66, the hardware configuration of the computer 78 will not be described here.

The external I/F 80 transmits and receives various types of information between one or more devices (hereinafter, also referred to as “second external devices”) outside the medical support device 24 and the processor 82.

As one of the second external devices, the control device 22 is connected to the external I/F 80. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic moving image 39 (see FIG. 1) from the processor 72 of the control device 22 via the external I/Fs 70 and 80 and performs various types of image processing on the acquired endoscopic moving image 39.

As one of the second external devices, the display device 18 is connected to the external I/F 80. The processor 82 controls the display device 18 via the external I/F 80 so that various types of information (for example, the endoscopic moving image 39 subjected to various types of image processing) are displayed on the display device 18.

Meanwhile, in recent years, a technology has been developed in which a trained model optimized by performing machine learning on a model (for example, a neural network) performs an object recognition process on each frame 40 included in the endoscopic moving image 39 to recognize the lesion 42, and a recognition result or information based on the recognition result is displayed on the screen 35.

However, in a stage of operating the trained model (that is, a stage in which the trained model is used in the endoscopy), in a case where an image that is not included in training data used in the machine learning (that is, an image in which an object that is not assumed in a learning stage is shown) is input to the trained model, the trained model may make erroneous recognition.

For example, the trained model may erroneously recognize an air bubble and/or a residue (for example, a residue caused by performing a medical treatment) shown in the frame 40 as the lesion 42, or may not recognize the lesion 42 even though the lesion 42 is present. In addition, a large difference between a composition of the frame 40 and a composition of the image used in machine learning (for example, a size of the lesion 42 shown in the frame 40 being too large or too small) may be one cause of erroneous recognition. In addition, a fact that an image quality of the frame 40 is too different from an image quality of the image used in machine learning can also be one cause of erroneous recognition. In the endoscopy, in a case where a result of erroneous recognition by the trained model is displayed on the screen 35, it is difficult for the doctor 12 to perform various medical treatments accurately.

As a method for resolving such erroneous recognition, a method of collecting a plurality of images that are highly likely to cause erroneous recognition and retraining the trained model by using the collected plurality of images is considered, but the retraining takes time and cost. In addition, a method of switching to a trained model trained with an image that is highly likely to cause erroneous recognition from an existing trained model can also be considered, but in this case, it takes time and cost for training and also takes time and cost for switching the trained model.

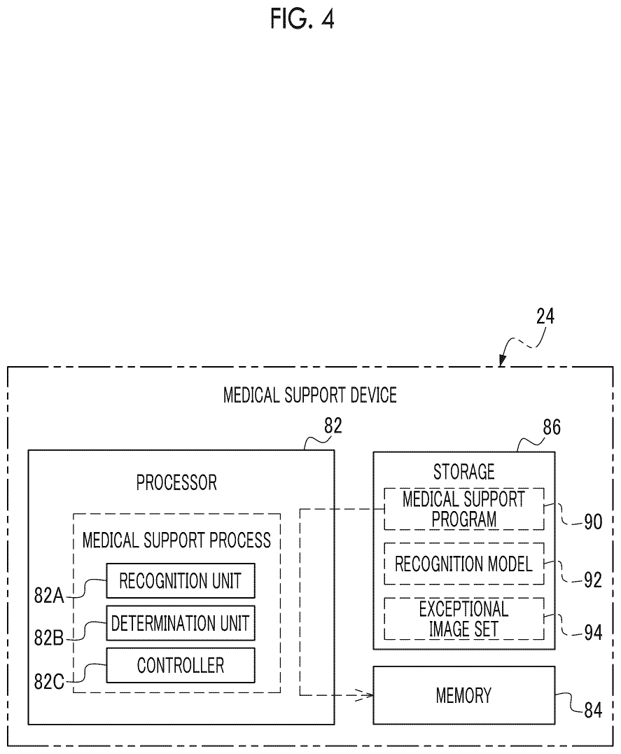

Therefore, in view of such circumstances, in the present embodiment, as shown in FIG. 4 as an example, the medical support process is performed by the processor 82 of the medical support device 24. FIG. 4 is a block diagram showing an example of functions of main units of the processor 82 included in the medical support device 24 and an example of information stored in the storage 86.

A medical support program 90 is stored in the storage 86. The medical support program 90 is an example of a “program” according to the present disclosure. The processor 82 reads out the medical support program 90 from the storage 86 and executes the read-out medical support program 90 on the memory 84 to perform the medical support process. The medical support process is realized by the processor 82 operating as a recognition unit 82A, a determination unit 82B, and a controller 82C according to the medical support program 90 executed on the memory 84.

The storage 86 stores a recognition model 92 and an exceptional image set 94. Although details will be described later, the recognition model 92 is used by the recognition unit 82A, and the exceptional image set 94 is used by the determination unit 82B. The frame 40 is input to the recognition model 92, and the recognition model 92 recognizes the lesion 42 shown in the input frame 40 and outputs a result of the recognition. In the present embodiment, the recognition model 92 is an example of a “trained model” according to the present disclosure.

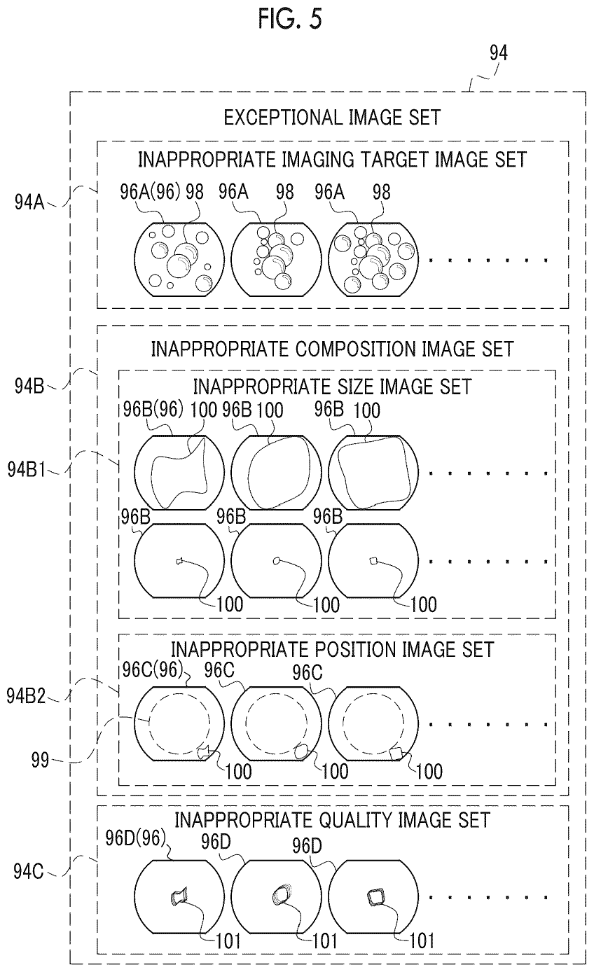

FIG. 5 is a conceptual diagram showing an example of a content of the exceptional image set 94. As shown in FIG. 5, the exceptional image set 94 includes a plurality of exceptional images 96 having different contents. The exceptional image 96 is an image determined as a medical image (here, as an example, an endoscopic image generated by imaging the inside of the large intestine 28 by the camera 52) that is inappropriate for use in the object recognition process (for example, a process in which the recognition model 92 recognizes the lesion 42). The medical image that is inappropriate for use in the object recognition process refers to, for example, a medical image in which a target object (for example, the lesion 42) is not recognized, or an object other than the target object and/or noise is erroneously recognized as the target object by the object recognition process. The exceptional image 96 may be an image generated by being captured by the camera or may be an image generated by generation AI. For example, a size and a shape of an outline of the exceptional image 96 are the same as a size and a shape of the frame 40. However, this is merely an example, and the technology of the present disclosure is established even in a case where the size and the shape of the outline of the exceptional image 96 are different from the size and the shape of the frame 40. In the present embodiment, the exceptional image 96 is an example of a “second medical image” and an “exceptional image” according to the present disclosure.

The exceptional image set 94 includes an inappropriate imaging target image set 94A, an inappropriate composition image set 94B, and an inappropriate quality image set 94C.

The inappropriate imaging target image set 94A includes a plurality of inappropriate imaging target images 96A in which the shown contents are different from each other. The concept of the exceptional image 96 includes the inappropriate imaging target image 96A. The inappropriate imaging target image 96A refers to an image in which an imaging target 98 that is inappropriate for use in the object recognition process (for example, a process in which the recognition model 92 recognizes the lesion 42) is shown (that is, an image in which an object that induces erroneous recognition of the object recognition process is shown). Examples of the inappropriate imaging target 98 include an air bubble and/or a residue. Examples of the residue include a residue generated by performing a medical treatment. The residue generated by the medical treatment refers to, for example, blood, a drug, a treatment scar, a marking region, a treatment tool, and/or smoke. Here, the imaging target 98 is an example of an “imaging target”, a “bubble”, and a “residue” according to the present disclosure.

The inappropriate composition image set 94B includes an inappropriate size image set 94B1 and an inappropriate position image set 94B2. The inappropriate size image set 94B1 includes a plurality of inappropriate size images 96B and a plurality of inappropriate position images 96C. Each of the plurality of inappropriate size images 96B and the plurality of inappropriate position images 96C is an image having a composition in which composition of a target object (for example, a lesion 100 in the example shown in FIG. 5) included in an imaging range of a camera used for imaging for obtaining the exceptional image 96 within the imaging range is inappropriate for use in the object recognition process (for example, a process in which the recognition model 92 recognizes the lesion 42) (that is, an image having a composition in which the target object (for example, the lesion 42) is not recognized, or an object other than the target object and/or noise is erroneously recognized as the target object by the object recognition process).

The contents shown in the plurality of inappropriate size images 96B are different from each other. The concept of the exceptional image 96 includes the inappropriate size image 96B. The inappropriate size image 96B refers to an image in which a size of a target object included in an imaging range of a camera used for imaging for obtaining the exceptional image 96 within the imaging range is excessively large or excessively small.

Examples of the image having a composition in which a size of a target object included in an imaging range of a camera used for imaging for obtaining the inappropriate size image 96B within the imaging range is excessively large include an image having a composition in which the size of the target object included in the imaging range within the imaging range is larger than a first size (for example, an image having a composition in which the size of the target object within the imaging range is large to the extent that the target object (for example, the lesion 42) is not recognized, or an object other than the target object and/or noise is erroneously recognized as the target object by the object recognition process). Examples of the first size include an upper limit value of a size range derived in advance by a test and/or a computer simulation or the like as a size range in which erroneous recognition is not caused in a case of being used for the object recognition process.

Examples of the image having a composition in which a size of a target object included in an imaging range of a camera used for imaging for obtaining the inappropriate size image 96B within the imaging range is excessively small include an image having a composition in which the size of the target object included in the imaging range within the imaging range is smaller than a second size (for example, an image having a composition in which the size of the target object within the imaging range is small to the extent that the target object (for example, the lesion 42) is not recognized or an object other than the target object and/or noise is erroneously recognized as the target object by the object recognition process). Examples of the second size include a lower limit value of a size range derived in advance by a test and/or a computer simulation or the like as a size range in which erroneous recognition is not caused in a case of being used for the object recognition process.

Here, the composition in which the size of the target object included in the imaging range within the imaging range is larger than the first size and the composition in which the size of the target object included in the imaging range within the imaging range is smaller than the second size are examples of a “first composition” according to the present disclosure.

The contents shown in the plurality of inappropriate position images 96C are different from each other. The concept of the exceptional image 96 includes the inappropriate position image 96C. The inappropriate position image 96C refers to an image having a composition in which a target object (the lesion 100 in the example shown in FIG. 5) included in an imaging range of a camera used for imaging for obtaining the exceptional image 96 deviates from a center region 99 of the imaging range. In a case where the target object included in the imaging range of the camera used for imaging for obtaining the exceptional image 96 deviates from the center region 99 of the imaging range, the target object is affected by an aberration of a lens. Therefore, there is a concern that the target object (for example, the lesion 42) is not recognized, or an object other than the target object and/or noise is erroneously recognized as the target object by the object recognition process. Therefore, here, a plurality of inappropriate position images 96C in which the shown contents are different from each other are prepared as the inappropriate position image set 94B2. Here, the composition in which the target object included in the imaging range of the camera used for imaging for obtaining the inappropriate position image 96C deviates from the center region 99 of the imaging range is an example of a “second composition” according to the present disclosure.

Here, as an example of the inappropriate composition, the composition in which the size of the target object within the imaging range is inappropriate and the composition in which the position of the target object within the imaging range is inappropriate have been illustrated, but this is merely an example. The inappropriate composition may be a composition in which an imaging direction with respect to the target object (that is, a direction in which the target object is shown in the image) is inappropriate. In addition, an image having a combined composition of two or more of a composition of an inappropriate size of the target object within the imaging range (that is, a size of the target object within the imaging range in which the target object (for example, the lesion 42) is not recognized, or an object other than the target object and/or noise is erroneously recognized as the target object by the object recognition process), a composition of an inappropriate position of the target object within the imaging range (that is, a position of the target object within the imaging range in which the target object (for example, the lesion 42) is not recognized, or an object other than the target object and/or noise is erroneously recognized as the target object by the object recognition process), and a composition of an inappropriate imaging direction with respect to the target object (that is, an imaging direction with respect to the target object within the imaging range in which the target object (for example, the lesion 42) is not recognized, or an object other than the target object and/or noise is erroneously recognized as the target object by the object recognition process) is used as the exceptional image 96.

The inappropriate quality image set 94C includes a plurality of inappropriate quality images 96D in which the shown contents are different from each other. The concept of the exceptional image 96 includes the inappropriate quality image 96D. The inappropriate quality image 96D refers to an image having an image quality that is inappropriate for use in the object recognition process (for example, a process in which the recognition model 92 recognizes the lesion 42) (for example, an image in which the target object (for example, the lesion 42) is not recognized, or an object other than the target object and/or noise is erroneously recognized as the target object by the object recognition process). Examples of the inappropriate image quality include out-of-focus and/or motion blur. Examples of the image quality of the inappropriate quality image 96D include an image quality derived in advance by a test and/or a computer simulation or the like as an image quality causing erroneous recognition in a case of being used for the object recognition process. Here, although the out-of-focus and/or the motion blur is illustrated as an example of the inappropriate image quality, this is merely an example. The inappropriate image quality may be an artifact, a ghost, a flare, a whiteout, and/or a black crush.

Here, for convenience of description, the inappropriate imaging target image 96A, the inappropriate size image 96B, the inappropriate position image 96C, and the inappropriate quality image 96D are separately illustrated, but this is merely an example. The exceptional image 96 may be an image having contents obtained by combining at least two or more of the content shown in the inappropriate imaging target image 96A, the content shown in the inappropriate size image 96B, the content shown in the inappropriate position image 96C, and the content shown in the inappropriate quality image 96D.

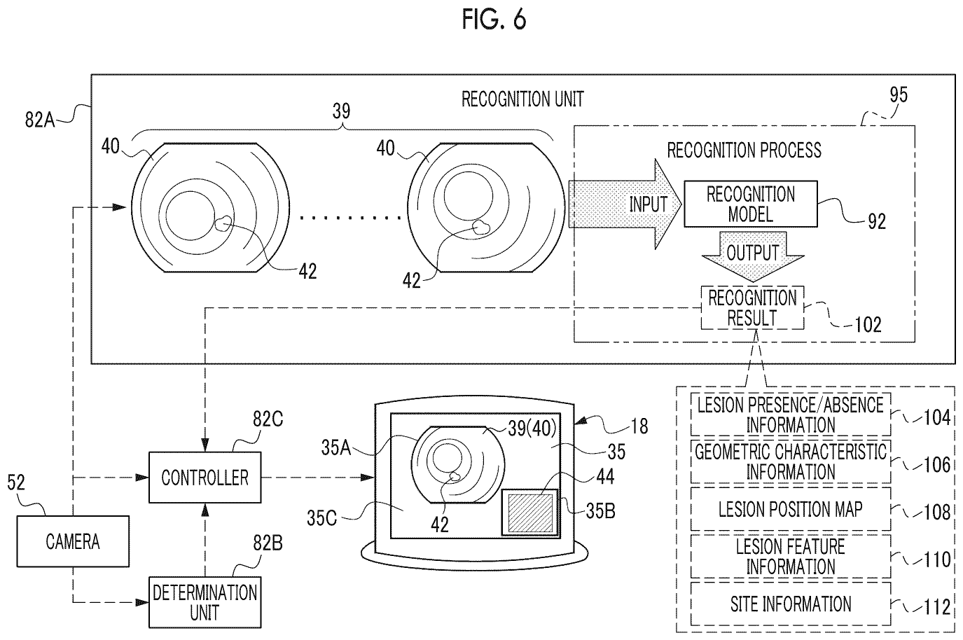

FIG. 6 is a conceptual diagram showing an example of processing contents of the recognition unit 82A and an example of an aspect around the recognition unit 82A. As shown in FIG. 6, the recognition unit 82A, the determination unit 82B, and the controller 82C acquire each of the plurality of frames 40 along the time series included in the endoscopic moving image 39, which is generated by being captured by the camera 52 at an imaging frame rate (for example, several tens of frames/second), from the camera 52 in units of one frame along the time series. Timings at which the recognition unit 82A, the determination unit 82B, and the controller 82C acquire the frame 40 are synchronized.

The controller 82C outputs the endoscopic moving image 39 to the display device 18. For example, the controller 82C displays the endoscopic moving image 39 in the first display region 35A as a live view image. That is, each time the frame 40 is acquired from the camera 52, the controller 82C displays the acquired frame 40 in the first display region 35A in order at a display frame rate (for example, several tens of frames/second). In addition, the controller 82C displays the assistance information 44 in the second display region 35B. In addition, the controller 82C updates the display content (for example, the assistance information 44) of the second display region 35B according to the display content of the first display region 35A.

The recognition unit 82A recognizes the lesion 42 in the endoscopic moving image 39 based on the endoscopic moving image 39 acquired from the camera 52. That is, the recognition unit 82A recognizes the lesion 42 shown in the frame 40 by sequentially performing a recognition process 95, which is an example of the object recognition process described above, on each of the plurality of frames 40 along the time series included in the endoscopic moving image 39 acquired from the camera 52.

For example, the recognition unit 82A recognizes geometric characteristics (for example, a position, a size, and a shape) of the lesion 42, a kind of the lesion 42, a type of the lesion 42 (for example, a pedunculated type, a subpedunculated type, a sessile type, a surface raised type, a surface flat type, a surface depressed type, and the like), and the like. Further, the recognition unit 82A recognizes a site in the endoscopic moving image 39 (hereinafter, simply referred to as a “site”) based on the endoscopic moving image 39 acquired from the camera 52. Here, the site refers to a site of the large intestine 28. Examples of the site of the large intestine 28 include an anal canal, a lower rectum, an upper rectum, a sigmoid colon, a descending colon, a transverse colon, an ascending colon, and an ileocecal portion.

The recognition process 95 is performed on the acquired frame 40 each time the frame 40 is acquired by the recognition unit 82A. The recognition process 95 is a process of recognizing the lesion 42 and the site by a method using AI. Here, as the recognition process 95, a process using the recognition model 92 is performed. The recognition process 95 is an example of an “object recognition process” according to the present disclosure.

The recognition model 92 is a trained model for object recognition in a bounding box method using AI. The recognition model 92 is optimized by performing machine learning on a neural network using training data. The training data is a data set including a plurality of data (that is, a plurality of frames of data) in which example data and correct answer data are associated with each other.

The example data is an image assuming the frame 40. First examples of the image assuming the frame 40 include an image obtained by actually imaging the inside of the large intestine with the camera. Second examples of the image assuming the frame 40 include an image virtually created (for example, an image generated by generation AI). The correct answer data is correct answer data (that is, an annotation) for the example data. Here, as an example of the correct answer data, an annotation for specifying geometric characteristics of a lesion shown in an image used as the example data, a kind of the lesion, a type of the lesion, and a site is used.

The recognition unit 82A acquires the frame 40 from the camera 52 and inputs the acquired frame 40 to the recognition model 92. As a result, the recognition model 92 recognizes the lesion 42 and the site shown in the input frame 40 each time the frame 40 is input, and generates and outputs a recognition result 102. In the present embodiment, the recognition result 102 is an example of a “processing result” according to the present disclosure.

The recognition unit 82A outputs the recognition result 102 itself to the display device 18 or the like in synchronization with the display of the frame 40 in the first display region 35A, or outputs information generated based on the recognition result 102 to the display device 18 or the like in synchronization with the display of the frame 40 in the first display region 35A. On the screen 35 (for example, the first display region 35A, the second display region 35B, and/or the third display region 35C) of the display device 18, the recognition result 102 itself is displayed, or the information generated based on the recognition result 102 is displayed. The display of the recognition result 102 itself and the display of the information generated based on the recognition result 102 are updated in synchronization with a display timing of the endoscopic moving image 39 displayed in the first display region 35A. That is, the display of the recognition result 102 itself and the display of the information generated based on the recognition result 102 are updated according to a display frame rate applied to the first display region 35A.

The recognition result 102 includes lesion presence/absence information 104. The lesion presence/absence information 104 is information indicating whether or not the lesion 42 is shown in the frame 40 input to the recognition model 92. In addition, the recognition result 102 includes geometric characteristic information 106, a lesion position map 108, lesion feature information 110, and the like.

The geometric characteristic information 106 is information (for example, coordinates) for specifying a shape, a size, and a position of the lesion 42 in the frame 40. The geometric characteristic information 106 or the information based on the geometric characteristic information 106 may be displayed in the first display region 35A, may be displayed in the second display region 35B as one of pieces of the assistance information 44, or may be displayed in the third display region 35C.

The lesion position map 108 is a map for specifying the position of the lesion 42 in the frame 40. The lesion feature information 110 is information for specifying the kind of the lesion 42 shown in the frame 40 input to the recognition model 92 and the type of the lesion 42.

Geometric characteristics (for example, a shape and a size of an outer contour) of the lesion position map 108 correspond to geometric characteristics (for example, a shape and a size of an outer contour) of the frame 40. The lesion position map 108 is displayed on the screen 35 by the controller 82C. In the present embodiment, the lesion position map 108 is displayed in the second display region 35B by the controller 82C as one of pieces of assistance information 44. For example, the display and non-display of the lesion position map 108 are switched according to the instruction received by the reception device 64 and/or various conditions. The lesion position map 108 displayed on the screen 35 is updated according to the display frame rate applied to the first display region 35A. With such a configuration, the doctor 12 can ascertain an approximate position of the lesion 42 in the endoscopic moving image 39 displayed in the first display region 35A by referring to the lesion position map 108 displayed in the second display region 35B while observing the endoscopic moving image 39 displayed in the first display region 35A.

The controller 82C displays the lesion feature information 110 on the screen 35. For example, the lesion feature information 110 may be displayed in the second display region 35B as one of pieces of the assistance information 44, or may be displayed in the first display region 35A and/or the third display region 35C.

The recognition result 102 also includes site information 112. The site information 112 is information for specifying a site shown in the frame 40. The site information 112 may be displayed in the first display region 35A, may be displayed in the second display region 35B as one of pieces of the assistance information 44, or may be displayed in the third display region 35C.

The determination unit 82B determines whether or not the display based on the recognition result 102 is to be performed based on the frame 40 acquired from the camera 52, and outputs a determination result to the controller 82C. The controller 82C performs or does not perform the display based on the recognition result 102 on the display device 18 based on the determination result input from the determination unit 82B.

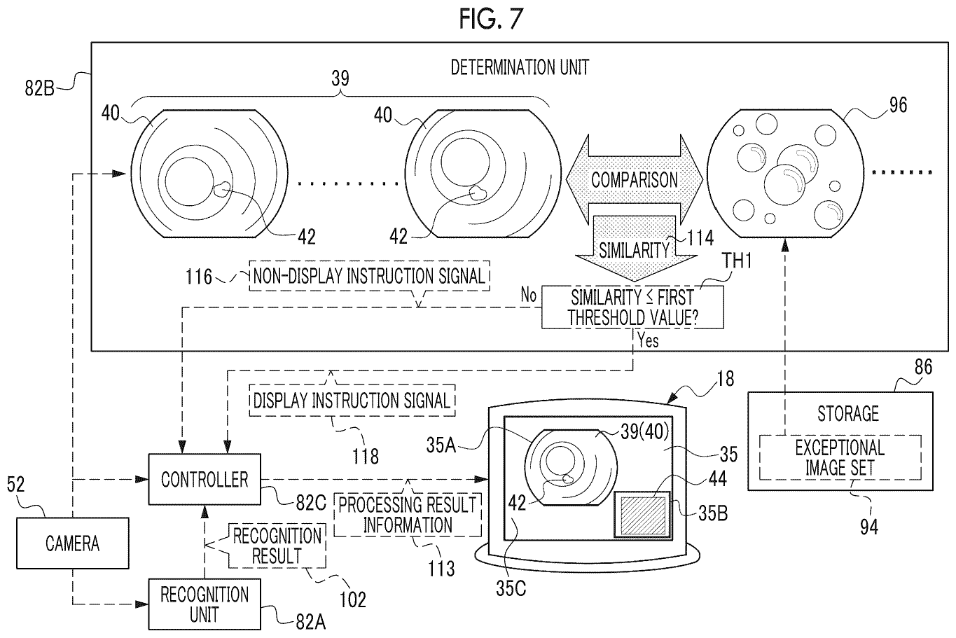

FIG. 7 is a conceptual diagram showing an example of processing contents of the determination unit 82B and an example of an aspect around the determination unit 82B. As shown in FIG. 7, the controller 82C acquires the recognition result 102 from the recognition unit 82A, and controls the display device 18 such that processing result information 113, which is information related to the acquired recognition result 102, is displayed on the screen 35. The processing result information 113 includes, for example, information for specifying the lesion 42, information indicating characteristics of the lesion 42, and/or information for specifying a site where the lesion 42 is present.

The determination unit 82B acquires all the exceptional images 96 from the exceptional image set 94 stored in the storage 86 each time the frame 40 is acquired from the camera 52. Moreover, the determination unit 82B calculates a similarity 114 between the frame 40 and the exceptional image 96 for each of the plurality of exceptional images 96 by comparing the frame 40 acquired from the camera 52 with each of all the exceptional images 96 acquired from the exceptional image set 94. In the present embodiment, the similarity 114 is an example of a “similarity” and a “comparison result” according to the present disclosure.

Here, for convenience of description, although a form example is described in which the frame 40 is compared with each of all the exceptional images 96 acquired from the exceptional image set 94, this is merely an example, and a comparison target with the frame 40 may not be all the exceptional images 96 acquired from the exceptional image set 94. The comparison target with the frame 40 may be a plurality of exceptional images 96 selected from the exceptional image set 94 according to the instruction received by the reception device 64 and/or various conditions (for example, a plurality of exceptional images 96 included in the inappropriate imaging target image set 94A, the inappropriate size image set 94B1, the inappropriate position image set 94B2, and/or the inappropriate quality image set 94C).

The similarity 114 is a degree to which the frame 40 and the exceptional image 96 are similar. For example, in a case where the frame 40 and the exceptional image 96 are completely matched, the similarity 114 indicates the highest value (for example, 100%), and the similarity 114 decreases as a difference between the frame 40 and the exceptional image 96 increases. The smaller the similarity 114 is, the higher the reliability of the recognition result 102 obtained by performing the recognition process 95 on the frame 40 used to calculate the similarity 114 (that is, the frame 40 compared with the exceptional image 96). The higher the similarity 114 is, the lower the reliability of the recognition result 102 is.

Therefore, the controller 82C makes a display aspect of the processing result information 113 different according to the similarity 114 calculated by the determination unit 82B. Hereinafter, this will be described in more detail.

In a case where all the similarities 114 calculated by comparing the frame 40 acquired from the camera 52 with all the exceptional images 96 acquired from the exceptional image set 94 exceed a first threshold value TH1 (for example, 80%), the determination unit 82B outputs a non-display instruction signal 116 to the controller 82C. The non-display instruction signal 116 is a signal for providing an instruction to non-display the processing result information 113. The first threshold value TH1 may be a fixed value or may be a variable value (for example, a value changed according to the instruction received by the reception device 64 and/or various conditions). The first threshold value TH1 is an example of a “first threshold value” according to the present disclosure.

On the other hand, in a case where all the similarities 114 calculated by comparing the frame 40 acquired from the camera 52 with all the exceptional images 96 acquired from the exceptional image set 94 are equal to or less than the first threshold value TH1, the determination unit 82B outputs a display instruction signal 118 to the controller 82C. The display instruction signal 118 is a signal for providing an instruction to display the processing result information 113.

The controller 82C displays the processing result information 113 in a first display aspect or a second display aspect according to the similarity 114. The first display aspect is a display aspect capable of specifying that the similarity 114 exceeds the first threshold value TH1. In the first display aspect, the processing result information 113 is not displayed. The second display aspect is a display aspect having higher visibility than the first display aspect and capable of specifying that the similarity 114 is equal to or less than the first threshold value TH1. In the second display aspect, the processing result information 113 is displayed.

That is, in a case where the non-display instruction signal 116 is input from the determination unit 82B, the controller 82C does not display the processing result information 113 on the screen 35, and in a case where the display instruction signal 118 is input from the determination unit 82B, the controller 82C displays the processing result information 113 on the screen 35.

The first display aspect described here is an example of a “first display aspect” according to the present disclosure, and the second display aspect described here is an example of a “second display aspect” according to the present disclosure.

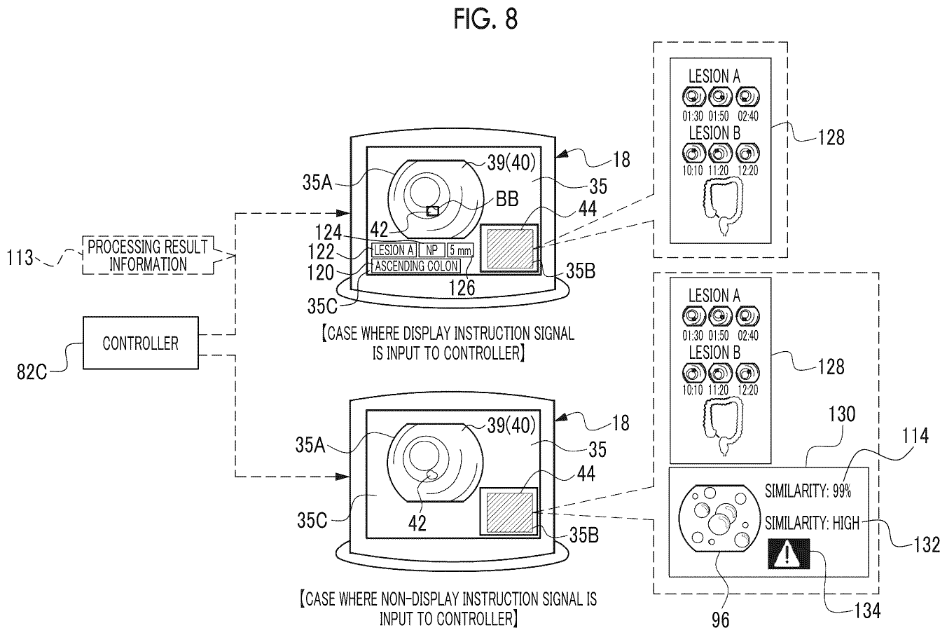

FIG. 8 is a conceptual diagram showing a screen example in a case where the processing result information 113 is displayed on the screen 35 and a screen example in a case where the processing result information 113 is not displayed on the screen 35. As shown in FIG. 8, in a case where the display instruction signal 118 is input from the determination unit 82B, the controller 82C generates site name information 120, lesion identification information 122, discrimination information 124, and size information 126 as the processing result information 113, and displays the generated information in the third display region 35C (in the example shown in FIG. 8, a region below the first display region 35A).

The site name information 120 is information indicating a name of a site recognized by performing the recognition process 95, and is generated based on the site information 112 (see FIG. 6). Here, although the site information 112 is illustrated, information other than the site information 112 may be adopted as long as the information can specify a site where the lesion 42 is present.

The lesion identification information 122 is information for identifying the lesion 42 recognized by performing the recognition process 95. The discrimination information 124 is information indicating a result (for example, a malignancy) of discriminating the lesion 42, and is generated based on the lesion feature information 110 (see FIG. 6). The size information 126 is information indicating a size of the lesion 42 recognized by performing the recognition process 95, and is generated based on the geometric characteristic information 106 (see FIG. 6). Here, although the lesion feature information 110 and the size information 126 are illustrated, information other than the lesion feature information 110 and the size information 126 may be adopted as long as the information indicates the characteristics of the lesion 42.

The lesion position map 108 (see FIG. 6) includes a bounding box BB, and in a case where the display instruction signal 118 is input from the determination unit 82B, the controller 82C displays the bounding box BB in the first display region 35A as the processing result information 113. The bounding box BB is a rectangular frame (for example, a rectangular border circumscribing an image region showing the lesion 42) for specifying a position recognized by the recognition model 92 as the position of the lesion 42 in the frame 40, which is shown in the frame 40. In the example shown in FIG. 8, the bounding box BB is displayed to be superimposed at a position where the lesion 42 in the frame 40 displayed in the first display region 35A is shown. The display of the bounding box BB is updated in synchronization with a display timing of the endoscopic moving image 39 displayed in the first display region 35A.

Here, the bounding box BB has been described, but this is merely an example. The controller 82C may display an identifier (for example, a mark defined at four corners of a rectangular frame) instead of the bounding box BB on the screen 35 (in the example shown in FIG. 8, the first display region 35A in the screen 35).

In the example shown in FIG. 8, the bounding box BB and the lesion identification information 122 are examples of “feature region information” according to the present disclosure, the site name information 120 is an example of “site specifying information” according to the present disclosure, and the discrimination information 124 and the size information 126 are examples of “characteristic information” according to the present disclosure.

In the example shown in FIG. 8, the controller 82C generates the past information 128 as one of pieces of the assistance information 44 and displays the past information 128 in the second display region 35B. The past information 128 is information generated based on the recognition result 102 obtained in the past. For example, the past information 128 includes various types of information displayed in the third display region 35C in the past, and an image, text, or the like that can specify an unobserved location or an untreated location in the current endoscopy.

On the other hand, in a case where the non-display instruction signal 116 is input from the determination unit 82B, the controller 82C does not display the processing result information 113 on the screen 35, and generates the past information 128 and similarity-related information 130 as one of pieces of the assistance information 44 to display the generated information in the second display region 35B. The similarity-related information 130 is information related to the similarity 114. Here, the similarity-related information 130 is an example of “similarity-related information” according to the present disclosure.

The similarity-related information 130 is information used in a determination process (see FIG. 7) by the determination unit 82B, the determination result by the determination unit 82B, and/or information generated based on the determination result by the determination unit 82B. In the example shown in FIG. 8, the similarity-related information 130 includes the exceptional image 96 used to calculate the similarity 114 exceeding the first threshold value TH1, the similarity 114 calculated by the determination unit 82B (that is, the similarity 114 exceeding the first threshold value TH1), similarity level information 132 indicating whether or not the similarity 114 exceeds the first threshold value TH1, and a notification mark 134 for notifying that the processing result information 113 is not displayed.

Here, the exceptional image 96 included in the similarity-related information 130 is an example of a “second medical image used to obtain the similarity” according to the present disclosure, and the similarity level information 132 and the notification mark 134 included in the similarity-related information 130 are examples of “information based on the similarity” according to the present disclosure.

In the example shown in FIG. 8, although a form example has been described in which the exceptional image 96 is included in the similarity-related information 130, this is merely an example. The similarity-related information 130 may include an image based on the exceptional image 96 used to calculate the similarity 114 exceeding the first threshold value TH1. Examples of the image based on the exceptional image 96 include an image obtained by processing the exceptional image 96 such as an image in which the exceptional image 96 is made into a thumbnail image or an image in which the exceptional image 96 is made into an achromatic image.

In the example shown in FIG. 8, although a form example is shown in which information corresponding to the similarity-related information 130 is not displayed on the screen 35 in a case where the similarity 114 is equal to or less than the first threshold value TH1, the information corresponding to the similarity-related information 130 may be displayed on the screen 35 (for example, the second display region 35B) in a case where the similarity 114 is equal to or less than the first threshold value TH1. Examples of the information corresponding to the similarity-related information 130 include the exceptional image 96 used to calculate the similarity 114 equal to or less than the first threshold value TH1, the similarity 114 calculated by the determination unit 82B (that is, the similarity 114 equal to or less than the first threshold value TH1), and/or information including the similarity level information 132.

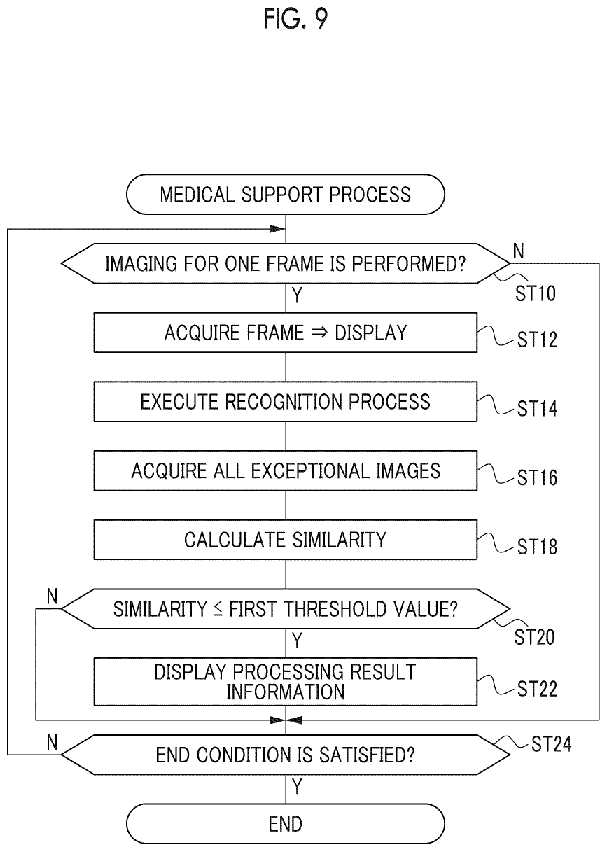

Next, an operation of a part of the endoscope apparatus 10 according to the present disclosure will be described with reference to FIG. 9. A flow of the medical support process shown in FIG. 9 is an example of a “medical support method” according to the present disclosure.

In the medical support process shown in FIG. 9, first, in step ST10, the recognition unit 82A determines whether or not imaging for one frame is performed by the camera 52 in the large intestine 28. In step ST10, in a case where the imaging for one frame is not performed by the camera 52 in the large intestine 28, a negative determination is made, and the medical support process proceeds to step ST24. In step ST10, in a case where the imaging for one frame is performed by the camera 52 in the large intestine 28, a positive determination is made, and the medical support process proceeds to step ST12.

In step ST12, the recognition unit 82A, the determination unit 82B, and the controller 82C acquire the frame 40 obtained by imaging the inside of the large intestine 28 (for example, the intestinal wall 32) by the camera 52. The controller 82C displays the frame 40 in the first display region 35A. In a case where the frame 40 is already displayed in the first display region 35A, the controller 82C updates the frame 40 displayed in the first display region 35A. That is, by repeatedly executing the process in step ST12, the frame 40 is displayed in the first display region 35A by the live view method. After the process in step ST12 is executed, the medical support process proceeds to step ST14.

In step ST14, the recognition unit 82A executes the recognition process 95 on the frame 40 acquired in step ST12. After the process in step ST14 is executed, the medical support process proceeds to step ST16.

In step ST16, the determination unit 82B acquires all the exceptional images 96 from the exceptional image set 94 stored in the storage 86. After the process in step ST16 is executed, the medical support process proceeds to step ST18.

In step ST18, the determination unit 82B calculates the similarity 114 for each of all the exceptional images 96 by comparing the frame 40 acquired in step ST12 with each of all the exceptional images 96 acquired in step ST16. After the process in step ST18 is executed, the medical support process proceeds to step ST20.

In step ST20, the determination unit 82B determines whether or not all the calculated similarities 114 are equal to or less than the first threshold value TH1. In step ST20, in a case where all the similarities 114 are equal to or less than the first threshold value TH1, a positive determination is made, and the medical support process proceeds to step ST22. In step ST20, in a case where all the similarities 114 are not equal to or less than the first threshold value TH1, a negative determination is made, and the medical support process proceeds to step ST24.

In step ST22, the controller 82C displays the processing result information 113 on the screen 35. After the process in step ST22 is executed, the medical support process proceeds to step ST24.

In step ST24, the controller 82C determines whether or not a medical support process end condition is satisfied. An example of the medical support process end condition is a condition that an instruction for the endoscope apparatus 10 to end the medical support process is given (for example, a condition that the reception device 64 receives an instruction to end the medical support process).

In a case where the medical support process end condition is not satisfied in step ST24, a negative determination is made, and the medical support process proceeds to step ST10. In a case where the medical support process end condition is satisfied in step ST24, a positive determination is made, and the medical support process ends.

As described above, in the endoscope apparatus 10 according to the present embodiment, the processing result information 113, which is information related to the recognition result 102 by the recognition process 95 performed on the frame 40 generated by being captured by the camera 52 inserted into the large intestine 28, is displayed on the screen 35 of the display device 18. In the endoscope apparatus 10, the similarity 114 between the frame 40 and the exceptional image 96 stored in the storage 86 is calculated. The exceptional image 96 is an image determined as a medical image that is inappropriate for use in the object recognition process 95, that is, a medical image in which the lesion 42 is not recognized, or an object other than the lesion 42 and/or noise is erroneously recognized as the lesion 42 by the recognition process 95.

In the endoscope apparatus 10, the display aspect of the processing result information 113 is different depending on the similarity 114. The lower the similarity 114, the higher the reliability of the recognition result 102. This means that the lower the similarity 114, the higher the reliability of the processing result information 113 displayed on the screen 35. Therefore, the doctor 12 can determine whether or not the processing result information 113 displayed on the screen 35 is reliable information from the display aspect of the processing result information 113. As a result, it is possible to prevent the doctor 12 from recognizing the inappropriate recognition result 102 by the recognition process 95 performed on the frame 40.

In addition, in the endoscope apparatus 10 according to the present embodiment, in a case where the similarity 114 exceeds the first threshold value TH1, the processing result information 113 is displayed on the screen 35 in the first display aspect capable of specifying that the similarity 114 exceeds the first threshold value TH1. In addition, in a case where the similarity 114 is equal to or less than the first threshold value TH1, the processing result information 113 is displayed on the screen 35 in the second display aspect capable of specifying that the similarity 114 is equal to or less than the first threshold value TH1. As a result, the doctor 12 can visually ascertain whether or not the similarity 114 exceeds the first threshold value TH1. In addition, the second display aspect has higher visibility than the first display aspect. For example, in the second display aspect, the processing result information 113 is displayed on the screen 35, and in the first display aspect, the processing result information 113 is not displayed on the screen 35. Therefore, as compared with a case where the processing result information 113 is displayed on the screen 35 in the same display aspect regardless of whether or not the similarity 114 is equal to or less than the first threshold value TH1, it is possible to make it easy for the doctor 12 to visually recognize that the similarity 114 is equal to or less than the first threshold value TH1. As a result, it is possible to make the doctor 12 visually recognize the highly reliable processing result information 113.

In addition, in the endoscope apparatus 10 according to the present embodiment, a medical image that is inappropriate for use in the recognition process 95 is used as the exceptional image 96. Then, in a case where the similarity 114 between the frame 40 and the exceptional image 96 exceeds the first threshold value TH1, the processing result information 113 is not displayed on the screen 35, and in a case where the similarity 114 between the frame 40 and the exceptional image 96 is equal to or less than the first threshold value TH1, the processing result information 113 is displayed on the screen 35. Therefore, it is possible to prevent the doctor 12 from making a medical determination based on the recognition result 102 obtained by performing the recognition process 95 on the frame 40 that is inappropriate for use in the recognition process 95.

In addition, in the endoscope apparatus 10 according to the present embodiment, the inappropriate imaging target image 96A is used as one of the exceptional images 96. Therefore, it is possible to prevent the doctor 12 from making a medical determination based on the recognition result 102 obtained by performing the recognition process 95 on the frame 40 in which the imaging target 98 (for example, an air bubble and/or a residue) that is inappropriate for use in the recognition process 95 is shown.

In addition, in the endoscope apparatus 10 according to the present embodiment, the inappropriate size image 96B and the inappropriate position image 96C are used as one of the exceptional images 96. Therefore, it is possible to prevent the doctor 12 from making a medical determination based on the recognition result 102 obtained by performing the recognition process 95 on the frame 40 having a composition that is inappropriate for use in the recognition process 95 (for example, a composition in which the size of the lesion 42 included in the imaging range within the imaging range is excessively large, a composition in which the size of the lesion 42 included in the imaging range within the imaging range is excessively small, and/or a composition in which the lesion 42 included in the imaging range deviates from the center region (that is, a region corresponding to the center region 99 shown in FIG. 5) of the imaging range).

In addition, in the endoscope apparatus 10 according to the present embodiment, the inappropriate quality image 96D is used as one of the exceptional images 96. Therefore, it is possible to prevent the doctor 12 from making a medical determination based on the recognition result 102 obtained by performing the recognition process 95 on the frame 40 having an image quality that is inappropriate for use in the recognition process 95 (for example, the frame 40 having out-of-focus and/or motion blur).

In addition, in the endoscope apparatus 10 according to the present embodiment, in a case where the similarity 114 is equal to or less than the first threshold value TH1, the site name information 120 is displayed in the third display region 35C as the information for specifying a site where the lesion 42 is present. Further, in a case where the similarity 114 is equal to or less than the first threshold value TH1, the bounding box BB and the lesion identification information 122 are displayed in the third display region 35C as the information for specifying the lesion 42. Further, in a case where the similarity 114 is equal to or less than the first threshold value TH1, the discrimination information 124 and the size information 126 are displayed in the third display region 35C as the information for specifying the characteristics of the lesion 42.

Therefore, a degree to which the doctor 12 perceives the site name information 120, the bounding box BB, the lesion identification information 122, the discrimination information 124, and the size information 126 can be made different according to the similarity 114. That is, in a case where the inappropriate recognition result 102 is obtained by the recognition process 95 performed on the frame 40 (for example, in a case where the similarity 114 exceeds the first threshold value TH1), the site name information 120, the bounding box BB, the lesion identification information 122, the discrimination information 124, and the size information 126 can be made difficult to be perceived by the doctor 12, and in a case where the appropriate recognition result 102 is obtained by the recognition process 95 performed on the frame 40 (for example, in a case where the similarity 114 is equal to or less than the first threshold value TH1), the site name information 120, the bounding box BB, the lesion identification information 122, the discrimination information 124, and the size information 126 can be made easy to be perceived by the doctor 12.

Further, in the present embodiment, in a case where the similarity 114 exceeds the first threshold value TH1, similarity-related information 130 (for example, information including the exceptional image 96, the similarity 114, the similarity level information 132, and the notification mark 134) is displayed in the second display region 35B. As a result, the doctor 12 can visually ascertain the similarity-related information 130 (for example, information including the exceptional image 96, the similarity 114, the similarity level information 132, and the notification mark 134). As a result, the doctor 12 can understand the reason why the processing result information 113 is not displayed on the screen 35.

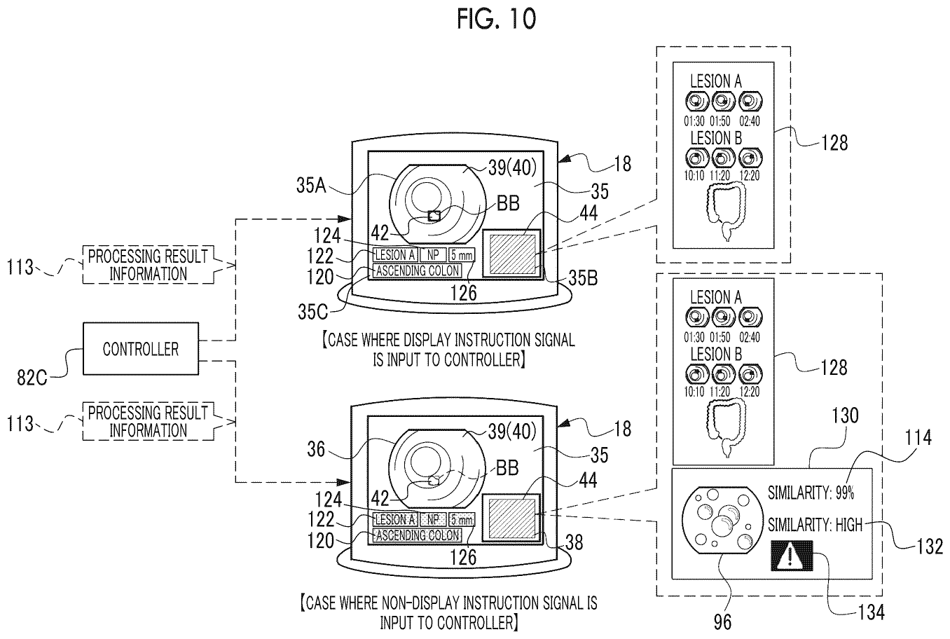

In the embodiment described above, although a form example has been described in which the processing result information 113 is not displayed on the screen 35 in a case where the similarity 114 exceeds the first threshold value TH1, this is merely an example. For example, as shown in FIG. 10, even in a case where the similarity 114 exceeds the first threshold value TH1 (that is, in a case where the non-display instruction signal 116 is input to the controller 82C), the processing result information 113 may be displayed on the screen 35. In this case, the visibility of the processing result information 113 is reduced. That is, in a case where the similarity 114 exceeds the first threshold value TH1, the visibility of the processing result information 113 displayed on the screen 35 is lower than that in a case where the similarity 114 is equal to or less than the first threshold value TH1 (that is, in a case where the display instruction signal 118 is input to the controller 82C). In the example shown in FIG. 10, the site name information 120, the lesion identification information 122, the discrimination information 124, and the size information 126 are displayed in the third display region 35C in a grayed-out state, and a line of the bounding BB is displayed to be thinner than a line of the bounding BB displayed in a case where the similarity 114 is equal to or less than the first threshold value TH1. There are various methods for reducing the visibility. For example, there are a method of reducing visibility by increasing transparency of the display, a method of reducing visibility by covering a display location with some kind of mask, and a method of reducing visibility by blinking.

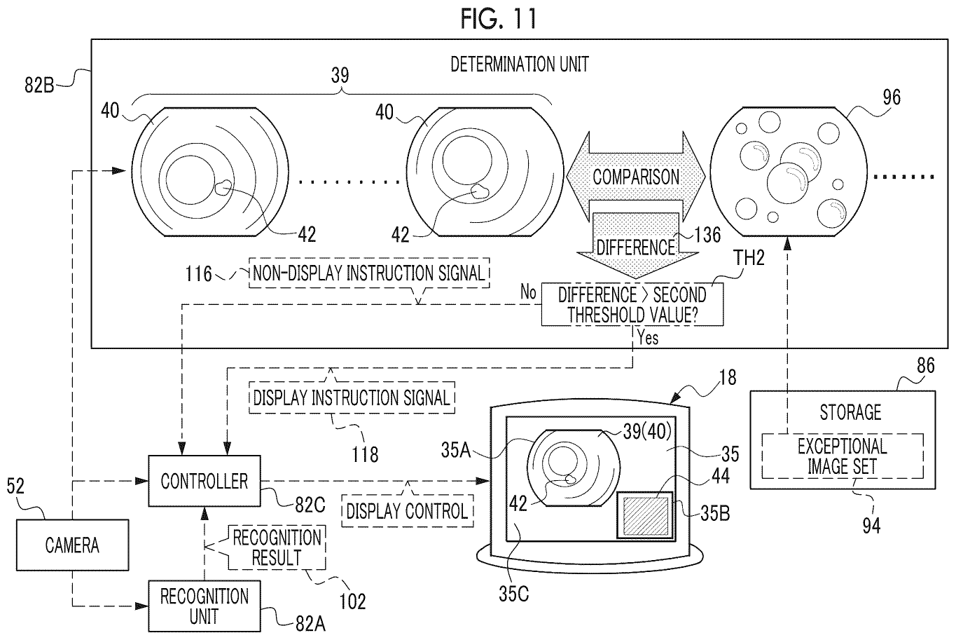

In the embodiment described above, a form example has been described in which the similarity 114 is calculated by the determination unit 82B, but this is merely an example. For example, as shown in FIG. 11, the determination unit 82B may calculate a difference 136, and the controller 82C may make the display aspect of the processing result information 113 different according to the difference 136. The difference 136 refers to a degree to which the frame 40 and the exceptional image 96 are different from each other. The determination unit 82B determines whether or not the difference 136 exceeds a second threshold value TH2.

Here, in a case where all the differences 136 calculated by comparing the frame 40 acquired from the camera 52 with all the exceptional images 96 acquired from the exceptional image set 94 are equal to or less than the second threshold value TH2 (for example, 80%), the determination unit 82B outputs the non-display instruction signal 116 to the controller 82C.

The second threshold value TH2 may be a fixed value or may be a variable value (for example, a value changed according to the instruction received by the reception device 64 and/or various conditions).

On the other hand, in a case where all the differences 136 calculated by comparing the frame 40 acquired from the camera 52 with all the exceptional images 96 acquired from the exceptional image set 94 exceed the second threshold value TH2, the determination unit 82B outputs the display instruction signal 118 to the controller 82C.

The controller 82C displays the processing result information 113 in a third display aspect or a fourth display aspect according to the difference 136. The third display aspect is a display aspect capable of specifying that the difference 136 equal to or less than the second threshold value TH2. In the third display aspect, the processing result information 113 is not displayed. The fourth display aspect is a display aspect having higher visibility than the third display aspect and capable of specifying that the difference 136 exceeds the second threshold value TH2. In the fourth display aspect, the processing result information 113 is displayed. In this way, the same effect as that of the embodiment described above can be obtained.

In the example shown in FIG. 11, the difference 136 is an example of a “difference” and a “comparison result” according to the present disclosure, and the second threshold value TH2 is an example of a “second threshold value” according to the present disclosure. In addition, the third display aspect described here is an example of a “first display aspect” and a “third display aspect” according to the present disclosure, and the fourth display aspect described here is an example of a “second display aspect” and a “fourth display aspect” according to the present disclosure.

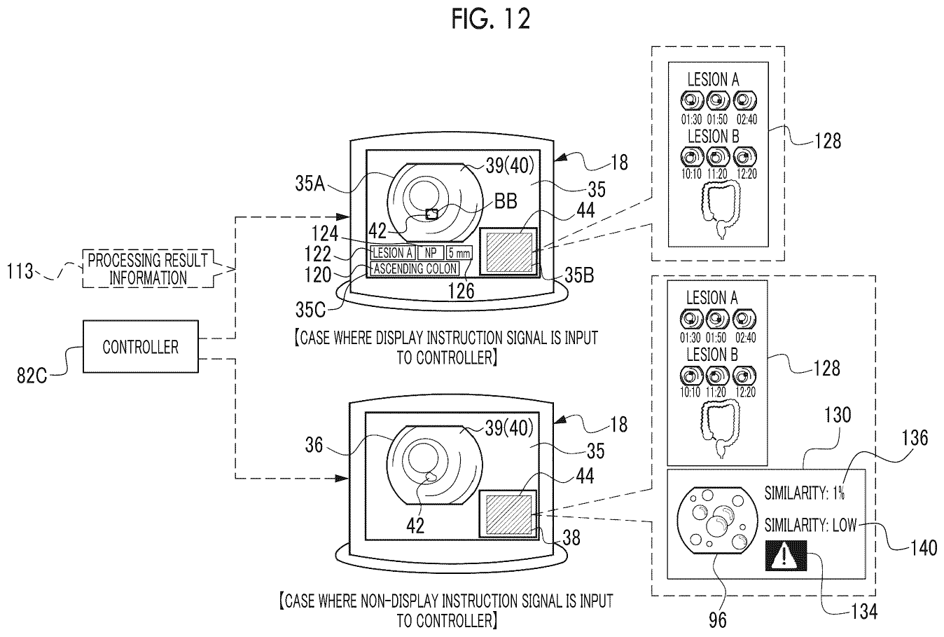

In addition, in the embodiment described above, a form example has been described in which, in a case where the similarity 114 exceeds the first threshold value TH1, the similarity-related information 130 is displayed in the second display region 35B. However, as shown in FIG. 12 as an example, in a case where the difference 136 is equal to or less than the second threshold value TH2 (that is, in a case where the non-display instruction signal 116 is input to the controller 82C), difference-related information 138, which is information related to the difference 136, may be displayed in the second display region 35B as one of pieces of the assistance information 44. Here, the difference-related information 138 is an example of “difference-related information” according to the present disclosure.

In the example shown in FIG. 12, the difference-related information 138 includes the exceptional image 96 used to calculate the difference 136 equal to or less than the second threshold value TH2, the difference 136 calculated by the determination unit 82B (that is, the difference 136 equal to or less than the second threshold value TH2), difference level information 140 indicating whether or not the difference 136 exceeds the second threshold value TH2, and the notification mark 134. By displaying the difference-related information 138 on the screen 35 in this way, the doctor 12 can visually ascertain the difference-related information 138. As a result, the doctor 12 can understand the reason why the processing result information 113 is not displayed on the screen 35.

The exceptional image 96 included in the difference-related information 138 is an example of a “second medical image used to obtain the difference” according to the present disclosure, and the notification mark 134 and the difference level information 140 included in the difference-related information 138 are examples of “information based on the difference” according to the present disclosure.

As described above, in the example shown in FIG. 12, in a case where the difference 136 is equal to or less than the second threshold value TH2, the difference-related information 138 (for example, information including the exceptional image 96, the difference 136, the difference level information 140, and the notification mark 134) is displayed in the second display region 35B. As a result, the doctor 12 can visually ascertain the difference-related information 138 (for example, information including the exceptional image 96, the difference 136, the difference level information 140, and the notification mark 134). As a result, the doctor 12 can understand the reason why the processing result information 113 is not displayed on the screen 35.

In the example shown in FIG. 12, although a form example has been described in which the exceptional image 96 is included in the difference-related information 138, this is merely an example. The difference-related information 138 may include an image based on the exceptional image 96 used to calculate the difference 136 equal to or less than the second threshold value TH2.

In the example shown in FIG. 12, although a form example is shown in which information corresponding to the difference-related information 138 is not displayed on the screen 35 in a case where the difference 136 exceeds the second threshold value TH2, the information corresponding to the difference-related information 138 may be displayed on the screen 35 (for example, the second display region 35B) in a case where the difference 136 exceeds the second threshold value TH2. Examples of the information corresponding to the difference-related information 138 include the exceptional image 96 used to calculate the difference 136 exceeding the second threshold value TH2, the difference 136 calculated by the determination unit 82B (that is, the difference 136 exceeding the second threshold value TH2), and/or information including the difference level information 140.

In the embodiment described above, although a form example has been described in which the determination unit 82B calculates the similarity 114 between each of all the exceptional images 96 acquired from the exceptional image set 94 and the frame 40 and compares all the similarities 114 with the first threshold value TH1, this is merely an example.

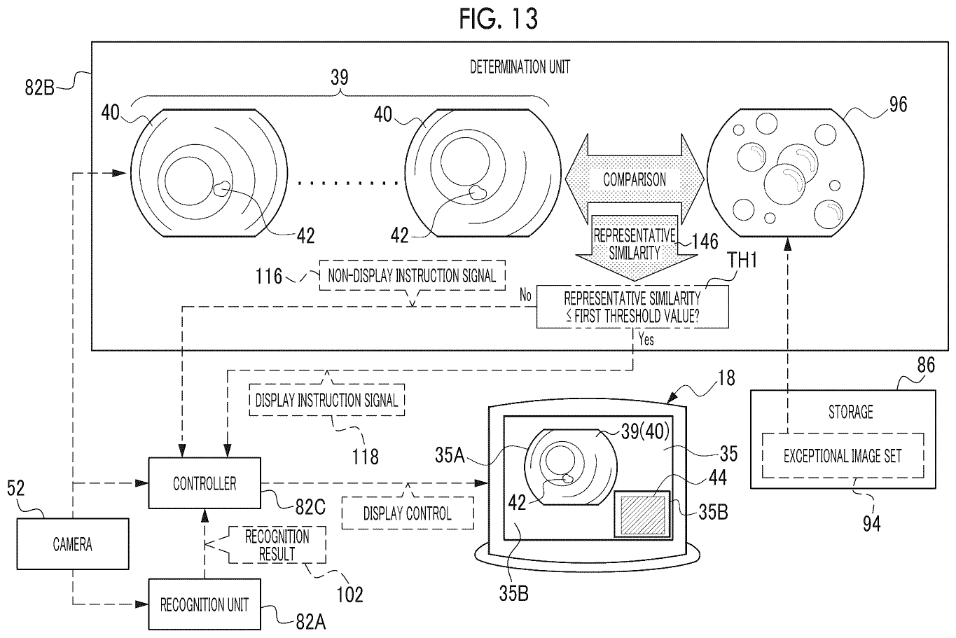

For example, as shown in FIG. 13, the determination unit 82B may calculate the similarity 114 between each of all the exceptional images 96 acquired from the exceptional image set 94 and the frame 40 and may compare a representative similarity 146, which is a similarity representative of a plurality of similarities 114 obtained by calculation (here, as an example, all the similarities 114), with the first threshold value TH1. The representative similarity 146 is an example of a “representative value” according to the present disclosure. Examples of the representative similarity 146 include a statistical value of the plurality of similarities 114. Examples of the statistical value include an average value, a maximum value, a minimum value, a median value, or a mode value. In this way, by comparing the representative similarity 146 with the first threshold value TH1, it is possible to reduce a processing load as compared with a case where the plurality of similarities 114 and the first threshold value TH1 are compared.

Here, the representative similarity 146 is illustrated, but this is merely an example. The determination unit 82B may calculate the difference 136 between each of all the exceptional images 96 acquired from the exceptional image set 94 and the frame 40, and may compare a representative difference, which is a difference representative of a plurality of differences 136 obtained by the calculation (here, as an example, all the differences 136), with the second threshold value TH2. In this case as well, the same effect can be expected.

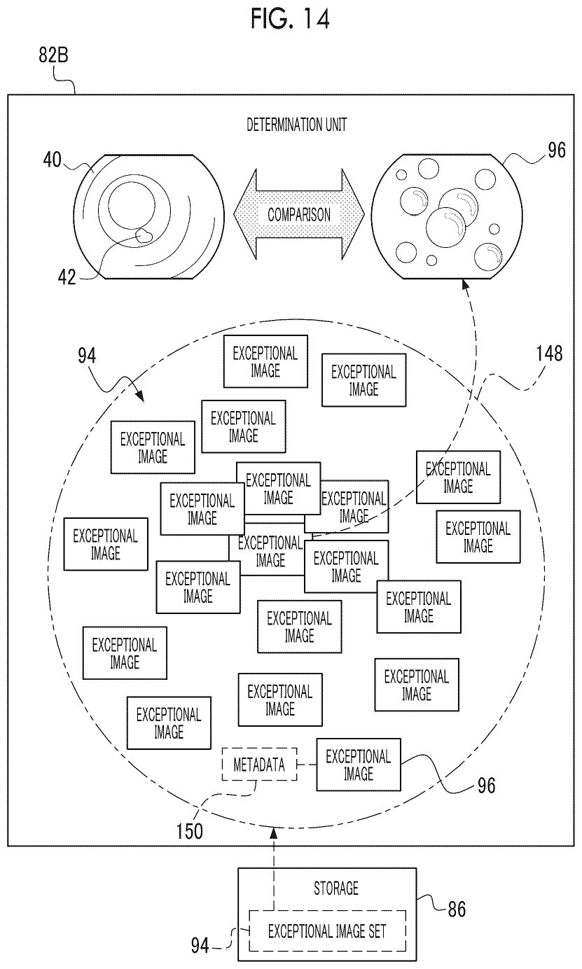

In addition, as shown in FIG. 14 as an example, the determination unit 82B may calculate the similarity 114 or the difference 136 by comparing the exceptional image 96 located at a centroid of a cluster 148 obtained by clustering a plurality of exceptional images 96 included in the exceptional image set 94 (here, as an example, all the exceptional images 96) with the frame 40.

Metadata 150 is added to each of the plurality of exceptional images 96. The metadata 150 includes a multidimensional feature quantity that defines the exceptional image 96, and the determination unit 82B clusters the plurality of exceptional images 96 by referring to the multidimensional feature quantity included in the metadata 150 of each exceptional image 96.

The multidimensional feature quantity included in the metadata 150 is a feature quantity related to a plurality of elements visually recognizable by a human, such as the number of pixels, a color, and a brightness of the exceptional image 96, and/or a feature quantity related to a plurality of elements visually unrecognizable by a human. For example, the multidimensional feature quantity is generated and output by a trained model (not shown) by inputting the exceptional image 96 to the trained model. Examples of the trained model used here include a trained model that generates and outputs a plurality of feature quantities by reducing a dimension of the input exceptional image 96. The multidimensional feature quantity included in the metadata 150 may be a multidimensional feature quantity extracted from a plurality of interlayers included in the recognition model 92 by inputting the exceptional image 96 to the recognition model 92.

As described above, by comparing the exceptional image 96 located at the centroid of the cluster 148 with the frame 40, it is possible to reduce the processing load as compared with a case where the plurality of similarities 114 and the first threshold value TH1 are compared. In addition, since the exceptional image 96 located at the centroid of the cluster 148 is compared with the frame 40, it is possible to reduce the processing load as compared with a case where the plurality of differences 136 are compared with the second threshold value TH2.

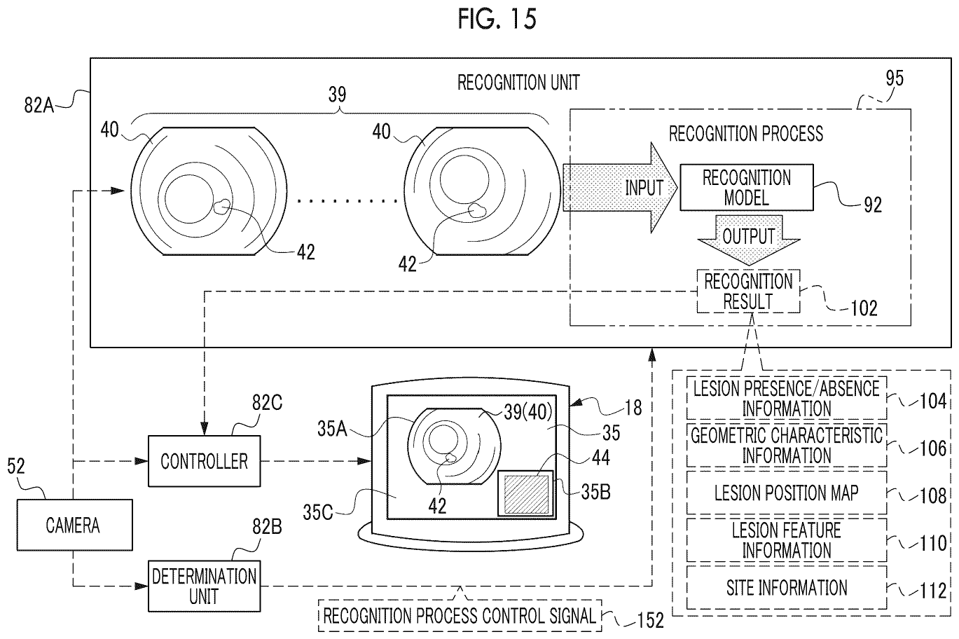

In the embodiment described above, a form example has been described on the premise that the recognition process 95 is executed by the recognition unit 82A regardless of the determination result by the determination unit 82B, but the present disclosure is not limited to this. For example, as shown in FIG. 15, the determination unit 82B may output a recognition process control signal 152 for controlling the recognition process 95 to the recognition unit 82A according to the determination result, and the recognition unit 82A may control the recognition process 95 according to the recognition process control signal 152.

For example, the determination unit 82B controls the recognition process 95 by outputting the recognition process control signal 152 to the recognition unit 82A such that the recognition process 95 is not executed on the frame 40 in a case where the similarity 114 exceeds the first threshold value TH1, and the recognition process 95 is executed on the frame 40 in a case where the similarity 114 is equal to or less than the first threshold value TH1. In this way, the same effect as that of the embodiment described above is obtained.

In addition, the determination unit 82B controls the recognition process 95 by outputting the recognition process control signal 152 to the recognition unit 82A such that the recognition process 95 is not executed on the frame 40 in a case where the difference 136 is equal to or less than the second threshold value TH2, and the recognition process 95 is executed on the frame 40 in a case where the difference 136 exceeds the second threshold value TH2. In this way, the same effect as that of the embodiment described above is obtained.

In the embodiment described above, the lesion 42 has been illustrated as an example of a “feature region” according to the present disclosure, but the present disclosure is not limited to this. Even in a case where a resection region, a bleeding region, a marking region, an organ, a treatment tool (for example, a hemostatic clip placed in the body), or the like is applied instead of the lesion 42, the technology of the present disclosure is established.

In the embodiment described above, although the camera 52 mounted on the endoscope 16 is illustrated, the present disclosure is not limited to this. For example, the technology of the present disclosure can be applied to a fundus camera, a camera mounted on a medical microscope, or the like.

In the embodiment described above, although a form example has been described in which the frame 40 is input to the recognition unit 82A and the processing result by the medical support process is output from the controller 82C, the present disclosure is not limited to this. For example, instruction data (so-called prompt) including the frame 40 may be input to so-called generation AI, and the processing result by the medical support process may be output from the generation AI. Examples of the generation AI include ChatGPT using GPT-4 (Internet search <https://openai.com/gpt-4>).

In addition, instruction data including at least a part of the processing result by the medical support process (for example, at least a part of the processing result information 113) may be used as the input information for the generation AI. Examples of the information output from the generation AI include information related to an operation content of the camera 52, information indicating a content of a medical treatment that is recommended to be performed during the endoscopy, and/or information indicating a content of a medical treatment that is recommended to be performed after the endoscopy. The information output from the generation AI may be stored in various storage regions (for example, the storage 76 and/or 86), displayed on the display device 18 as the assistance information 44, printed on a medium by the printer, or output from a speaker as a voice.

In the embodiment described above, the recognition process 95 using AI in a bounding box method has been illustrated, but this is merely an example. For example, a recognition process using AI in a segmentation method may be performed instead of the recognition process 95 using AI in a bounding box method. In addition, a recognition process in a non-AI method (for example, a template matching method) may be performed instead of the recognition process in an AI method, or a recognition process in which the non-AI method and the AI method are combined may be performed.

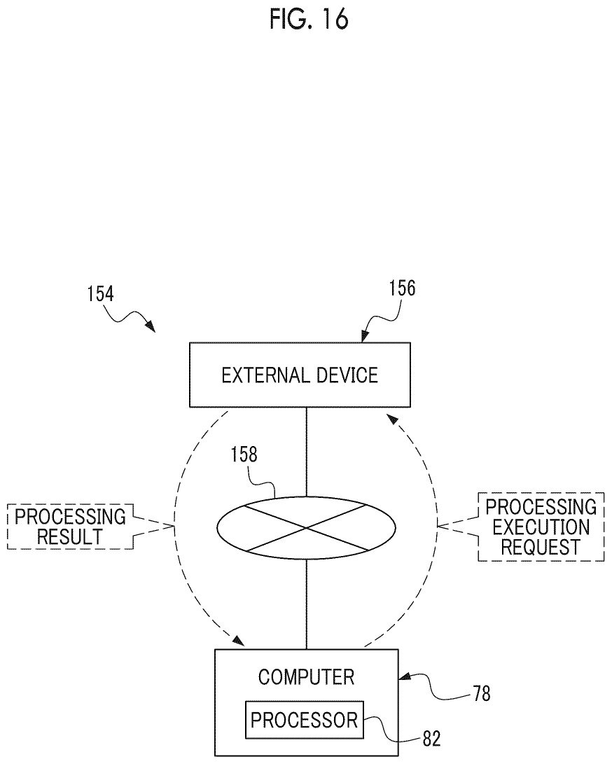

In the embodiment described above, a form example is described in which the medical support process is performed by the computer 78, but the present disclosure is not limited to this. At least some of processing included in the medical support process may be performed by a device provided outside the computer 78. Hereinafter, an example of this case will be described with reference to FIG. 16.

FIG. 16 is a conceptual diagram showing an example of a configuration of an endoscope apparatus 154. The endoscope apparatus 154 is an example of an “endoscope apparatus” according to the present disclosure. The endoscope apparatus 154 is different from the endoscope apparatus 10 described in the embodiment described above in that an external device 156 is provided.

The external device 156 is communicably connected to the computer 78 via a network 158 (for example, a WAN and/or a LAN).

Examples of the external device 156 include at least one server that directly or indirectly performs transmission and reception of data with the computer 78 via the network 158. The external device 156 receives a processing execution instruction given from the processor 82 of the computer 78 via the network 158. Then, the external device 156 executes processing according to the received processing execution instruction and transmits a processing result to the computer 78 via the network 158. In the computer 78, the processor 82 receives the processing result transmitted from the external device 156 via the network 158 and executes a process using the received processing result.

Examples of the processing execution instruction include an instruction for the external device 156 to execute at least a part of the medical support process. First examples of at least a part (that is, processing executed by the external device 156) of the medical support process include the recognition process 95. In this case, the external device 156 executes the recognition process 95 in response to the processing execution instruction given from the processor 82 via the network 158 and transmits the recognition result 102 to the computer 78 via the network 158. In the computer 78, the processor 82 receives the recognition result 102 and executes the same processing as in the above-described embodiment by using the received recognition result 102.

Second examples of at least a part of the medical support process (that is, processing executed by the external device 156) include processing by the determination unit 82B. In this case, the external device 156 executes the processing by the determination unit 82B in response to the processing execution instruction given from the processor 82 via the network 158, and transmits a processing result (for example, the non-display instruction signal 116, the display instruction signal 118, the similarity 114, the difference 136, the exceptional image 96 used to calculate the similarity 114, and/or the exceptional image 96 used to calculate the difference 136) to the computer 78 via the network 158. In the computer 78, the processor 82 receives the processing result and executes the same processing as in the above-described embodiment (for example, the display using the display device 18) by using the received processing result.

Third examples of at least a part of the medical support process (that is, processing executed by the external device 156) include processing by the controller 82C. In this case, the external device 156 executes processing by the controller 82C in response to the processing execution instruction given from the processor 82 via the network 158, and transmits a processing result (for example, at least a part of the processing result information 113) to the computer 78 via the network 158. In the computer 78, the processor 82 receives the processing result and executes the same processing as in the above-described embodiment (for example, the display using the display device 18) by using the received processing result.

For example, the external device 156 is realized by cloud computing. It should be noted that the cloud computing is merely an example, and the external device 156 may be realized by network computing such as fog computing, edge computing, or grid computing. Instead of the server, at least one personal computer or the like may be used as the external device 156. In addition, a computing device having a communication function equipped with a plurality of types of AI functions may be used.

In the embodiment described above, a form example is described in which the medical support program 90 is stored in the storage 86, but the present disclosure is not limited to this. For example, the medical support program 90 may be stored in a portable computer-readable non-transitory storage medium, such as an SSD or a USB memory. The medical support program 90 stored in the non-transitory storage medium is installed in the computer 78 of the endoscope apparatus 10. The processor 82 executes the medical support process according to the medical support program 90.

In addition, the medical support program 90 may be stored in a storage device of another computer, server, or the like connected to the endoscope apparatus 10 via a network, and the medical support program 90 may be downloaded and installed in the computer 78 in response to a request from the endoscope apparatus 10.

It is not necessary to store all the medical support programs 90 in a storage device of another computer, server device, or the like connected to the endoscope apparatus 10 or to store all the medical support programs 90 in the storage 86, and a part of the medical support programs 90 may be stored.

The following various processors can be used as hardware resources for executing the medical support process. Examples of the processor include a CPU which is a general-purpose processor that executes software, that is, a program, to function as the hardware resource executing the medical support process. In addition, examples of the processor include a dedicated electric circuit which is a processor having a circuit configuration specially designed for executing specific processing, such as an FPGA, a PLD, or an ASIC. A memory is incorporated in or connected to any processor, and any processor executes the medical support process using the memory.

The hardware resource for executing the medical support process may be configured by one of the various processors or by a combination of two or more processors of the same type or different types (for example, a combination of a plurality of FPGAs or a combination of a CPU and an FPGA). Further, the hardware resource for executing the medical support process may be one processor.

As an example of the configuration using one processor, first, there is a form in which one processor is configured by a combination of one or more CPUs and software, and the processor functions as the hardware resource for executing the medical support process. Second, as typified by a SoC or the like, there is a form in which a processor that realizes all functions of a system including a plurality of hardware resources executing the medical support process with one IC chip is used. As described above, the medical support process is realized using one or more of the various processors as the hardware resource.

Further, as a hardware structure of these various processors, more specifically, an electrical circuit in which circuit elements such as semiconductor elements are combined can be used. Further, the above-described medical support process is only an example. Therefore, it is needless to say that unnecessary steps may be deleted, new steps may be added, or a processing order may be changed without departing from the gist of the present disclosure.

The above-described contents and illustrated contents are detailed descriptions of parts related to the present disclosure, and are merely examples of the present disclosure. For example, the above descriptions related to configurations, functions, operations, and advantageous effects are descriptions related to examples of configurations, functions, operations, and advantageous effects of the parts related to the present disclosure. Therefore, it is needless to say that unnecessary parts may be deleted, or new elements may be added or replaced with respect to the above-described contents and illustrated contents without departing from the gist of the present disclosure. In order to avoid complications and easily understand the parts according to the present disclosure, in the above-described contents and illustrated contents, common technical knowledge and the like that do not need to be described to implement the present disclosure are not described.

All documents, patent applications, and technical standards described in the present specification are incorporated in the present specification by reference to the same extent as in a case where each document, patent application, and technical standard are specifically and individually noted to be incorporated by reference.

In regard to the embodiment described above, the following appendices will be further disclosed.

### Appendix 1

A medical support method comprising: acquiring a first medical image generated by being captured by a camera; and not executing an object recognition process on the first medical image in a case where a similarity between the first medical image and a second medical image stored in a storage exceeds a first threshold value TH1, and executing the object recognition process in a case where the similarity is equal to or less than the first threshold value TH1.

### Appendix 2

A medical support method comprising: acquiring a first medical image generated by being captured by a camera; and not executing an object recognition process on the first medical image in a case where a difference between the first medical image and a second medical image stored in a storage is equal to or less than a second threshold value TH2, and executing the object recognition process in a case where the difference exceeds the second threshold value TH2.

### Appendix 3

A program for causing a computer to execute a medical support process comprising: acquiring a first medical image generated by being captured by a camera; and not executing an object recognition process on the first medical image in a case where a similarity between the first medical image and a second medical image stored in a storage exceeds a first threshold value TH1, and executing the object recognition process in a case where the similarity is equal to or less than the first threshold value TH1.

### Appendix 4

A program for causing a computer to execute a medical support process comprising: acquiring a first medical image generated by being captured by a camera; and not executing an object recognition process on the first medical image in a case where a difference between the first medical image and a second medical image stored in a storage is equal to or less than a second threshold value TH2, and executing the object recognition process in a case where the difference exceeds the second threshold value TH2.

### Appendix 5

A non-transitory computer-readable storage medium storing a program for causing a computer to execute a medical support process comprising: acquiring a first medical image generated by being captured by a camera; and not executing an object recognition process on the first medical image in a case where a similarity between the first medical image and a second medical image stored in a storage exceeds a first threshold value TH1, and executing the object recognition process in a case where the similarity is equal to or less than the first threshold value TH1.

### Appendix 6

A non-transitory computer-readable storage medium storing a program for causing a computer to execute a medical support process comprising: acquiring a first medical image generated by being captured by a camera; and not executing an object recognition process on the first medical image in a case where a difference between the first medical image and a second medical image stored in a storage is equal to or less than a second threshold value TH2, and executing the object recognition process in a case where the difference exceeds the second threshold value TH2.

### Appendix 7

A medical support device comprising a processor, in which the processor is configured to: display, on a display device, processing result information which is information related to a processing result of an object recognition process performed on a first medical image generated by being captured by a camera; and make a display aspect of the processing result information different according to a similarity or a difference between the first medical image and a second medical image stored in a storage.

### Appendix 8

The medical support device according to Appendix 7, in which in a case where the difference is equal to or less than a second threshold value, the processing result information is displayed in a third display aspect capable of specifying that the difference is equal to or less than the second threshold value, and in a case where the difference exceeds the second threshold value, the processing result information is displayed in a fourth display aspect capable of specifying that the difference exceeds the second threshold value.

### Appendix 9

The medical support device according to Appendix 8, in which the fourth display aspect has higher visibility than the third display aspect.

### Appendix 10

The medical support device according to Appendix 9, in which the processing result information is not displayed in the third display state, and the processing result information is displayed in the fourth display state.

### Appendix 11

A medical support device comprising a processor, in which in a case where a difference between a first medical image generated by being captured by a camera and a second medical image stored in a storage is equal to or less than a second threshold value, the processor does not execute an object recognition process on a first medical image, and in a case where the difference exceeds the second threshold value, the processor executes the object recognition process.

What is claimed is:

1. A medical support device comprising a processor,
wherein the processor is configured to:
display, on a display device, processing result information which is information related to a processing result of an object recognition process performed on a first medical image generated by being captured by a camera; and
make a display aspect of the processing result information different according to a similarity or a difference between the first medical image and a second medical image stored in a storage.

2. The medical support device according to claim 1,
wherein in a case where the similarity exceeds a first threshold value or the difference is equal to or less than a second threshold value, the processing result information is displayed in a first display aspect capable of specifying that the similarity exceeds the first threshold value or the difference is equal to or less than the second threshold value, and
in a case where the similarity is equal to or less than the first threshold value or the difference exceeds the second threshold value, the processing result information is displayed in a second display aspect capable of specifying that the similarity is equal to or less than the first threshold value or the difference exceeds the second threshold value.

3. The medical support device according to claim 2,
wherein the second display aspect has higher visibility than the first display aspect.

4. The medical support device according to claim 3,
wherein the processing result information is not displayed in the first display aspect, and
the processing result information is displayed in the second display aspect.

5. The medical support device according to claim 1,
wherein the second medical image is an exceptional image determined as a medical image that is inappropriate for use in the object recognition process.

6. The medical support device according to claim 5,
wherein the exceptional image is an image in which an imaging target that is inappropriate for use in the object recognition process is shown.

7. The medical support device according to claim 6,
wherein the imaging target includes an air bubble and/or a residue.

8. The medical support device according to claim 5,
wherein the exceptional image is an image having a composition that is inappropriate for use in the object recognition process.

9. The medical support device according to claim 8,
wherein the composition is a first composition and/or a second composition,
the first composition is a composition in which a size of a target object included in an imaging range within the imaging range is larger than a first size or a composition in which the size within the imaging range is smaller than a second size, and
the second composition is a composition in which the target object deviates from a center region of the imaging range.

10. The medical support device according to claim 5,
wherein the exceptional image is an image having an image quality that is inappropriate for use in the object recognition process.

11. The medical support device according to claim 10,
wherein the image quality includes out-of-focus and/or motion blur.

12. The medical support device according to claim 1,
wherein the storage stores a plurality of the second medical images having different contents, and
the similarity or the difference is a representative value of a plurality of comparison results obtained by comparing the first medical image with each of the plurality of second medical images, or a comparison result obtained by comparing the second medical image located at a centroid of a cluster obtained by clustering the plurality of second medical images with the first medical image.

13. The medical support device according to claim 1,
wherein the processing result information includes feature region information for specifying a feature region recognized by the object recognition process, characteristic information indicating characteristics of the feature region, and/or site specifying information for specifying a site where the feature region is present.

14. The medical support device according to claim 1,
wherein the display device displays similarity-related information which is information related to the similarity, or difference-related information which is information related to the difference.

15. The medical support device according to claim 14,
wherein the similarity-related information includes the similarity, information based on the similarity, the second medical image used to obtain the similarity, and/or an image based on the second medical image used to obtain the similarity, and
the difference-related information includes the difference, information based on the difference, the second medical image used to obtain the difference, and/or an image based on the second medical image used to obtain the difference.

16. The medical support device according to claim 1,
wherein the camera is mounted on an endoscope and is inserted into a body to image an inside of the body.

17. The medical support device according to claim 1,
wherein the object recognition process is a process of recognizing a lesion.

18. The medical support device according to claim 1,
wherein the processing result is generated by a trained model by inputting the first medical image to the trained model.

19. A medical support device comprising a processor,
wherein in a case where a similarity between a first medical image generated by being captured by a camera and a second medical image stored in a storage exceeds a first threshold value or a difference between the first medical image and the second medical image is equal to or less than a second threshold value, the processor does not execute an object recognition process on a first medical image, and in a case where the similarity is equal to or less than the first threshold value or the difference exceeds the second threshold value, the processor executes the object recognition process.

20. An endoscope apparatus comprising:
the medical support device according to claim 1; and
the camera.

21. A medical support method comprising:
displaying, on a display device, processing result information which is information related to a processing result of an object recognition process performed on a first medical image generated by being captured by a camera; and
making a display aspect of the processing result information different according to a similarity or a difference between the first medical image and a second medical image stored in a storage.

22. A non-transitory computer-readable storage medium storing a program executable by a computer to execute a process comprising:
displaying, on a display device, processing result information which is information related to a processing result of an object recognition process performed on a first medical image generated by being captured by a camera; and
making a display aspect of the processing result information different according to a similarity or a difference between the first medical image and a second medical image stored in a storage.
